(19) United States
(12) Patent Application Publication
(10) Pub. No.: US 2025/0170172 A1
(43) Pub. Date: May 29, 2025

(54) METHODS FOR TREATING LENTIVIRUS INFECTION

(71) Applicant: Jericho Sciences, LLC, Durham, NC (US)

(72) Inventor: Heidi Kay, Chapel Hill, NC (US)

(21) Appl. No.: 18/841,860

(22) PCT Filed: Feb. 28, 2023
(86) PCT No.: PCT/US2023/014140

§ 371 Date: Aug. 27, 2024

Related U.S. Application Data
(60) Provisional application No. 63/315,040, filed on Feb. 28, 2022.

(51) Int. Cl. A61K 33/243; A61K 31/513; A61K 31/5383; A61K 31/675; A61K 31/685; A61P 31/20

(52) U.S. Cl. CPC A61K 33/243; A61K 31/513; A61K 31/5383; A61K 31/675; A61K 31/685; A61P 31/20

(57) ABSTRACT

The present invention relates to methods of treating lentivirus and other viral infections. Viral zinc finger domains are considered critical targets across multiple virus families, as they are highly mutationally restricted and serve critical roles in both virus replication and in virus-host interactions, which are the sabotaging interactions that can cause complications of inflammation and immune dysregulation. Human immunodeficiency virus mutates faster than any other known virus, establishing precedence for other potential virus infection indications.

This application is an International Application which claims priority from U.S. provisional patent application No. 63/315,040, filed on Feb. 28, 2022, the entire contents of which are incorporated herein by reference.

For countries that permit incorporation by reference, all patents, patent applications and publications cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted being prior art.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Small Business Innovation Research Grant No. MH09666 awarded by the National Institutes of Health. The government has certain rights in the invention.

### FIELD OF THE INVENTION

This invention is directed to compositions and methods for treating lentivirus and other viral infections.

### BACKGROUND OF THE INVENTION

Human immunodeficiency virus is a lentivirus causing a chronic, lifelong infection. The virus genome inserts into long-lived cells of the host, preventing clearance. While combination antiretroviral therapy (cART, each of which is an antiretroviral, or ARV), has significantly improved the health of individuals infected with human immunodeficiency virus-1 (HIV), available HIV antiviral therapeutics largely target virus replication pathways such that treatment interruption results in the rapid resumption of viral replication, continuing loss of CD4⁺ lymphocytes and compounding immunologic dysfunction. Despite successful therapeutic control of replication with the current standard of care (cART), chronic inflammation, HIV-associated neurocognitive disorders, cardiovascular disease and other comorbidities remain continuing challenges for an estimated 38 million people now living with HIV globally. While new guidelines recommending earlier initiation of cART may help control the loss of CD4⁺ T cells, consequences of morbidity and mortality, increasing drug resistance, long term consequences of cART toxicities, nonadherence to an effective cART regimen and long-term healthcare costs remain problematic for individuals and health care systems managing chronic HIV-1 infection. New therapeutic strategies that can provide longer-term virus suppression and reductions in sources of persistent virus are being sought to relieve drug burdens and comorbidities, and to improve the lives of people living with HIV infection (PLWH).

### SUMMARY OF THE INVENTION

Aspects of the invention relate to compositions and methods for treating lentivirus and other viral infections. For example, in embodiments, the invention provides a method to reduce persistent sources of virus in tissues, including virus in compartmental reservoirs.

For example, aspects of the invention are drawn towards a method of treating or preventing a viral infection, or alleviating a symptom thereof. In embodiments, the method comprises administering to a subject a compound according to Formula (1a) or Formula (1b)

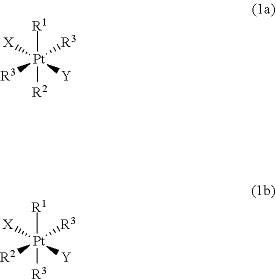

wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)₂ wherein R¹ is NO₂, COOH, COOR², COR, OH or SO₃H; wherein R² is F, Cl, Br, I, CN, SCN, NCS, NO₂, ONO, OHSO₂, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²), sulfate (SO₄, phosphate (HPO₄)²⁻, selenate (SeO₄)²⁻, or silicate (SiO₄)²⁻, an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxy carbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

In embodiments, X and Y are Cl, R¹ is NO₂, R² is Cl, and R³ is NH₃. This structure is referred to as “FX101” and/or “FX” in examples.

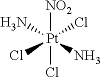

In embodiments, the orientation of X and Y are trans to one another.

In embodiments, the compound is selected from the group consisting of a compound according to Formula (1a) or Formula (1b)

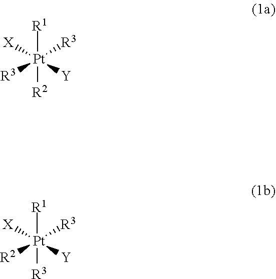

wherein X and Y are independently, nucleophilic exchangeable leaving groups; wherein R¹ is NO₂ or OH; wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)²⁻, phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

In embodiments, the virus comprises a lentivirus. For example, the lentivirus comprises HIV, SIV, SHIV, or FIV.

In embodiments, the virus is not HIV, SIV, SHIV, or FIV.

In embodiments, the virus comprises a Retrovirus, Coronavirus, Paramyxovirus, Togavirus, Flavivirus, Bunyavirus, or a Hepadnavirus.

In embodiments, the virus comprises an Oncoretrovirus, Human T-lymphotropic virus (HTLV), Feline lymphotropic virus (FeLV), Spumavirus, a Nidovirus, Severe Respiratory Syndrome Corona Virus 2 (SARS-CoV-2), Middle East Respiratory Syndrome (MERS-CoV), Severe Respiratory Syndrome Corona Virus 1 (SARS-CoV-1), a Henipavirus, Measles virus (MeV), Nipah virus (NiV), Mumps virus (MuV), Sendai virus (SeV), Parainfluenza virus 5 (PIV-5), Human parainfluenza virus (HPIV); Hendra virus (HeV), Newcastle Disease virus (NDV), an Alphavirus, Chikungunya virus (CHIKV), Sindbis virus (SINV), Dengue virus-1, Dengue virus-2, Dengue virus-3, Dengue Virus-4, West Nile virus (WNV), Japanese encephalitis virus (JEV), Zika virus, Yellow Fever Virus (YFV), a Nairovirus, Crimean Congo Hemorrhagic Fever (CCHF) virus, a Hantavirus, an Orthobunyavirus, an Arenavirus, or Hepatitis B virus (HBV).

Embodiments can further comprise administering to the subject at least one additional antiviral therapy.

In embodiments, the subject has previously been treated with at least one additional antiviral therapy. For example, the antiviral therapy comprises an antiretroviral therapy. For example, the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof. For example, the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1 or any combination thereof. For example, the antiviral therapy comprises a broadly neutralizing antibody. For example, the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

In embodiments, the antiviral therapy comprises combination antiretroviral therapy (cART).

In embodiments, treating or preventing a viral infection is indicated by reduced lentivirus levels in lymphatic tissue, reduced lentivirus in cerebrospinal fluid, increased IL-21 production in lymphatic tissue, development of broadly neutralizing antibodies, reduced viral setpoint, reduced frequency of provirus in peripheral blood mononuclear cells, or any combination thereof.

Further, aspects of the invention are drawn towards a method of treating or preventing a viral infection, or alleviating a symptom thereof by administering to a subject a first agent and a second agent, wherein the first agent comprises an antiviral therapy, and wherein the second agent comprises a compound according to Formula (1a) or Formula (1b)

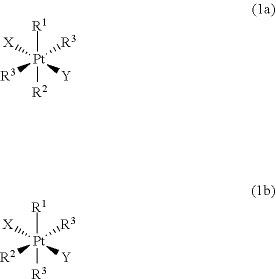

wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)², selenate (SeO₄)², or silicate (SiO₄)²; wherein R¹ is NO₂, COOH, COOR², OR, COR, OH or SO₃H; wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²), sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₅, OHSeO₂, N-alkyl, alkyl, alkoxy, cycloalkyl, cycloalkoxy, aryl, aryloxy, alkylcarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxy carbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

In embodiments, X and Y are Cl, R¹ is NO₂, R² is Cl, and R³ is NH₃.

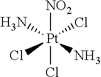

In embodiments, the orientation of X and Y are trans to one another.

In embodiments, the compound is selected from the group consisting of a compound according to Formula (1a) or Formula (1b),

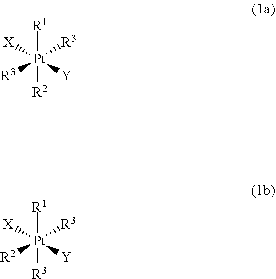

wherein X and Y are independently, nucleophilic exchangeable leaving groups; wherein R¹ is NO₂ or OH; wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)₂, phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

In embodiments, the first agent and the second agent can be administered simultaneously or sequentially.

In embodiments, the virus comprises a lentivirus. For example, the lentivirus comprises HIV, SIV, SHIV, or FIV.

In embodiments, the virus is not HIV, SIV, SHIV, or FIV.

In embodiments, the virus comprises a Retrovirus, Coronavirus, Paramyxovirus, Togavirus, Flavivirus, Bunyavirus, or a Hepadnavirus.

In embodiments, the virus comprises an Oncoretrovirus. Human T-lymphotropic virus (HTLV), Feline lymphotropic virus (FeLV), Spumavirus, a Nidovirus, Severe Respiratory Syndrome Corona Virus 2 (SARS-CoV-2), Middle East Respiratory Syndrome (MERS-CoV), Severe Respiratory Syndrome Corona Virus 1 (SARS-CoV-1), a Henipavirus, Measles virus (MeV), Nipah virus (NiV), Mumps virus (MuV), Sendai virus (SeV), Parainfluenza virus 5 (PIV-5), Human parainfluenza virus (HPIV); Hendra virus (HeV), Newcastle Disease virus (NDV), an Alphavirus, Chikungunya virus (CHIKV), Sindbis virus (SINV), Dengue virus-1, Dengue virus-2, Dengue virus-3, Dengue Virus-4, West Nile virus (WNV), Japanese encephalitis virus (JEV), Zika virus, Yellow Fever Virus (YFV), a Nairovirus, Crimean Congo Hemorrhagic Fever (CCHF) virus, a Hantavirus, an Orthobunyavirus, an Arenavirus, or Hepatitis B virus (HBV).

In embodiments, the antiretroviral therapy comprises combination antiretroviral therapy (cART).

For example, the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

For example, the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1, or any combination thereof.

For example, the antiviral therapy comprises a broadly neutralizing antibody.

For example, the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

In embodiments, treating or preventing a viral infection is indicated by reduced lentivirus levels in lymphatic tissue, reduced lentivirus in cerebrospinal fluid, increased IL-21 production in lymphatic tissue, development of broadly neutralizing antibodies, reduced viral setpoint, reduced frequency of provirus in peripheral blood mononuclear cells, or any combination thereof.

Still further, aspects of the invention are drawn towards an antiviral composition. In embodiments, the antiviral composition comprises a first agent and a second agent, wherein the first agent comprises an antiviral therapy, and wherein the second agent comprises a compound according to Formula (1a) or Formula (1b)

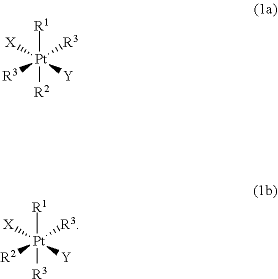

wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻; wherein R¹ is NO₂, COOH, COOR², OR, COR, OH or SO₃H; wherein R² is F, Cl, Br, I, CN, SCN, NCS, NO₂, ONO, OHSO₃, OH₂PO₃, OHSO₂, SO₃H, OH, OR², OS(CH)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²), sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkylcarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

The antiviral composition of claim 35, wherein X and Y are Cl, R¹ is NO₂, R² is Cl, and R³ is NH₃.

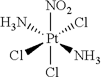

The antiviral composition of claim 35, wherein the orientation of X and Y are trans to one another.

The antiviral composition of claim 35, wherein the compound is selected from the group consisting of a compound according to Formula (1a) or Formula (1b),

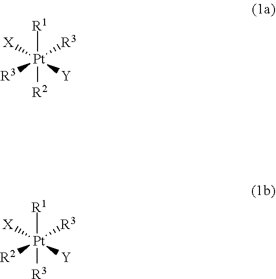

wherein X and Y are independently, nucleophilic exchangeable leaving groups; wherein R¹ is NO₂ or OH; wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂. NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

In embodiments, the antiretroviral therapy comprises combination antiretroviral therapy (cART).

For example, the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

For example, the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1, or any combination thereof.

For example, the antiviral therapy comprises a broadly neutralizing antibody.

For example, the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

Other objects and advantages of this invention will become readily apparent from the ensuing description.

### BRIEF DESCRIPTION OF THE FIGURES

FIG. 1 illustrates in vitro competitive binding of FX101 to the zinc binding domain of nucleocapsid protein peptide in the presence of zinc ions using Matrix-assisted laser desorption ionization (MALDI)—time of flight (TOF) analyses. Mixtures were prepared in equimolar concentrations of ˜2×10⁻⁴ Molar solutions in saline. The uppermost graph shows the peptide with FX101 and zinc (Pep+Fx+Zn). The second graph down shows the peptide mixed with FX101 (Pep+FX). The third graph down shows the peptide mixed with zinc (Pep+Zn) The bottom graph shows the peptide alone (Pep). These graphs show that FX101 binds to the peptide, even in the presence of zinc. Mass Spectrometer: Voyager DE STR (Applied Biosystems). Software: Voyager Instrument Control Panel (v 5.1) and Data Explorer (v4.0) Instrument Method: (a) ACTH_Reflector_Aug24_2010; Accelerating voltage 25000; Grid voltage 63.8%; Mirror voltage ratio1.12, Laser Rep Rate 20.0 Hz Band width 500: Vertical Scale 50, Bin size 0.5: Acquisition mass range 800-4000 Da; Delay 155, 165, or 200 sec, Laser Intensity 1480-1590. (b) angiotensine.bic, 75 nsec delay time Calibration: C1 or C2 standards in DHB matrix. Matrix: DHB (Laser BioLabs, France), 10 mg/ml in (a) 50% acetonitrile/50% water-0.1% TFA/50% water, no TFA, and (b) 50% ethanol/50% water, no TFA. Spotted: 0.5 μL sample.

FIG. 2 illustrates differences in group mean viral load setpoints between treatment groups post cART treatment cessation. Animals achieving any period of virus suppression (<50 RNA copies/mL) exhibited differences in viral load setpoints commencing approximately 16.5 weeks post cART cessation. The bar graph depicts the group means+/−standard error of the mean. The dotted line indicates the limit of detection (50 RNA copies/mL) for measuring viremia by this RT-PCR method.

FIG. 3 illustrates differences in individual median viral load setpoints between treatment groups post cART treatment cessation. Animals achieving any period of virus suppression (<50 RNA copies/mL) exhibited differences in viral load setpoints commencing approximately 16.5 weeks post cART cessation. The dot graph depicts individual animal measures for each group with the bar at the median. The dotted line indicates the limit of detection (50 RNA copies/mL) for measuring viremia by this RT-PCR method.

FIG. 4 illustrates that those animals not achieving a period of virus suppression exhibited higher viral setpoints and no differences between treatment groups. The bar graph depicts the group means+/−standard error of the mean. The dotted line indicates the limit of detection (50 RNA copies/mL) for measuring viremia by this RT-PCR method.

FIG. 5 illustrates those animals not achieving a period of virus suppression exhibited higher viral setpoints and no differences between treatment groups. The dot graph depicts individual animal measures for each group with the bar at the median. The dotted line indicates the limit of detection (50 RNA copies/mL) for measuring viremia by this RT-PCR method.

FIG. 6 illustrates the pretreatment (post infection) viremia-time area under the curve for the four treatment groups, showing that the FX+cART group had no selection advantages over the acute infection phase after infection (pretreatment) to anticipate long term reductions in viral setpoints post treatment. The four treatment groups are separated by spaces, with a mean pretreatment viremia-time area under the curve of 5.33×10⁷ RNA copies/mL-wks for the cART group, 9.69×10⁷ for the FX+cART group, 2.80×10⁷ for the FX group and 6.91×10⁶ for the control group. The numbers on the bars indicate the viremia-time area under the curve for that animal. An asterix (*) after the animal ID indicates animals achieving a subsequent period of virus suppression (between 2-8 weeks, “suppressed”).

FIG. 7 illustrates logarithmic dot plots (Base 2) of cerebrospinal fluid (CSF) viremia at indicated time points in the study timeline for all animals grouped by treatment, regardless of whether they achieved a period of plasma virus suppression (<50 RNA copies/mL). Bars indicate the median value for that treatment group.

FIG. 8 illustrates logarithmic (base 2) dot plots of cerebrospinal fluid (CSF) viremia at indicated time points in the study timeline for unsuppressed animals grouped by treatment. Bars indicate the median value for that treatment group.

FIG. 9 illustrates logarithmic (base 2) dot plots of cerebrospinal fluid (CSF) viremia at indicated time points in the study timeline for animals achieving a period of plasma virus suppression (<50 RNA copies/mL), grouped by treatment. Suppressed animals achieved a period of plasma viremia suppression between 2-8 weeks over the study timeline Bars indicate the median value for that treatment group.

FIG. 10 illustrates logarithmic (base 2) dot plots of cerebrospinal fluid (CSF) viremia at indicated time points in the study timeline for unsuppressed animals in the FX only and Control groups. None of these animals were virally suppressed (<50 RNA copies/mL) over the treatment period. Bars indicate the median value for that treatment group.

FIG. 11 illustrates selected soluble factors that were measured from cerebrospinal fluid (CSF) at indicated times over the study timeline using multiplex fluorescent beads (Magpix—Merck Millipore and Thermo Scientific). Some of these plots use logarithmic scales to capture the range of responses. Results for animals receiving cART (circles) versus FX+cART (square) indicate a relatively lower level of CNS inflammation at termination for animals in the FX+cART group, regardless of whether they achieved a period of undetectable virus suppression (<50 RNA copies/mL).

FIG. 12 illustrates a bar graph depicting the frequency of proviral DNA in peripheral blood mononuclear cells (PBMC) at termination for animals in the cART group (horizontal shaded bars), FX group (black bars) and FX+cART group (grey bars) as measured using droplet digital PCR. SIV positive counts were those which were double positive for both SIVenv and SIVpol sequences.

FIG. 13 illustrates the development of Tier 2 antibodies in eight cART-treated animals in this study. Two of the eight animals developed detectable Tier 2 antibodies. One animal (CG64), noted with an asterisk, did not produce detectable Tier 1 antibodies, was not virally suppressed at any time point, and is considered a rapid progressor. Excluding the rapid progressor, two of seven animals produced Tier 2 antibodies. Viral titers (ID50; Log base 2) were determined using serum collected at scheduled time points post cART cessation.

FIG. 14 illustrates the development of Tier 2 antibodies in ten animals receiving FX101 (“FX”) in this study. Six of the ten animals developed detectable Tier 2 antibodies. Two of these animals (BI14 and CC66) received FX only and were never virally suppressed. The other eight animals received FX+cART. Of these, two animals (BK48 and CC67), noted with an asterisk, did not produce significant Tier 1 antibodies, were not virally suppressed at any time point, and are considered rapid progressors. Excluding the rapid progressors, six of eight animals produced Tier 2 antibodies. Of the two animals not producing Tier 2 antibodies, BK79 was the lowest IL-21 producer of the entire study, possibly indicating a genetic factor contributing to this outcome. Animal BM24 was controlling viremia below detection for several weeks post treatment cessation, with low antigen levels possibly contributing to this outcome. Viral titers (ID50; Log base 2) were determined using serum collected at scheduled time points post cART cessation.

FIG. 15 illustrates a dot plot of the median frequency of IL-21 mRNA positive cells using FISH (fluorescent in situ hybridization) in lymph nodes for all cotherapeutically-treated animals in the FX+cART group (squares) was higher than for cART-treated animals (circles) at study termination (0.453 vs. 0.374, p=0.164, nonparametric) but not for spleen. Animals had been off cART for about 27 weeks at this time point, so this measure reflects a sustained immunologic reconstitution in the lymph nodes. The outlier in the FX+cART group (lowest point on graph) had the lowest CD4⁺ T cell count, indicating that this animal may not have had sufficient T cells to generate IL-21. Interestingly, IL-21 mRNA frequencies in the FX-only treated animals (up triangles) were notably higher than for the control animals (down triangles) in both lymph node and spleen.

FIG. 16 illustrates a Pearson correlation of SIV positive cell frequencies with IL-21 positive cell frequencies, as measured by FISH, for animals achieving a period of plasma virus suppression (<50 RNA copies/mL). The line graph illustrates a significant relationship in lymph node tissues (p=0.047 Pearson r=−0.6080; 95% confidence, two-tailed R²=0.3697). The squares show cART-treated animals, and the circles show FX+cART-treated animals.

FIG. 17 illustrates a dot plot of comparative treatment group frequencies of SIV mRNA positive cells per million cells at study termination, as determined using FISH. These frequencies in both spleen and lymph node tissues were found to be lower in the FX+cART group (squares) as compared with the cART group (circles) for animals achieving a period of plasma virus suppression (<50 RNA copies/mL). Bars indicate the median value for that treatment group. The outlier in the FX+cART group with the highest SIV frequencies did not produce Tier 2 antibodies, did not produce high levels of IL-21 and measured very low frequencies of CD4⁺ T cells, characteristic of a rapid progressor.

FIG. 18 illustrates a dot plot of comparative treatment group frequencies of SIV mRNA positive cells per million cells at study termination, as determined using FISH. These frequencies in spleen were found to be lower in the FX+cART group (squares) as compared with the cART group (circles) for animals regardless of whether they achieved a period of plasma virus suppression (<50 RNA copies/mL). Bars indicate the median value for that treatment group.

FIG. 19 illustrates a dot plot of comparative treatment group frequencies of SIV mRNA positive cells per million cells at study termination, as determined using FISH. These frequencies in lymph nodes were found to be lower in the FX+cART group (squares) as compared with the cART group (circles) for animals regardless of whether they achieved a period of plasma virus suppression (<50 RNA copies/mL). Bars indicate the median value for that treatment group. The outlier in the FX+cART group with the highest SIV frequencies did not produce Tier 2 antibodies, did not produce high levels of IL-21 and measured very low frequencies of CD4⁺ T cells, characteristic of a rapid progressor. In this graph, the outlier is particularly distinct.

FIG. 20 illustrates flow cytometric analysis of whole blood that was collected at 25 Weeks post infection (10 weeks no treatment, 15 weeks of drug treatment) from eight animals in the first Cohort, including two animals from each treatment group. One hundred microliters of whole blood were labeled using fluorescent antibodies for CD3⁺, CD4⁺ and CD8⁺ markers to identify T cell subpopulations. The relative gated percent of CD3′CD4⁺ T cells (left bar in each pair) shows that cART cotherapy with FX101 (FX+cART) comparatively prevents loss of CD4⁺ T cells versus FX101 alone (FX), further benefiting animals that achieved a period of virus suppression (“Sup”, <50 RNA copies/mL). The black line depicts the CD4⁺:CD8⁺ T cell ratios, with values noted.

FIG. 21 illustrates flow cytometric analysis of whole blood that was collected at 36 Weeks post infection (10 weeks no treatment, 15 weeks of drug treatment followed by 11 weeks post treatment cessation) from seven animals in the first Cohort, including two animals in each treatment group less one animal in the FX group that was terminated due to a secondary infection (common complication of SIV infection). One hundred microliters of whole blood were labeled using fluorescent antibodies for CD3⁺, CD4⁺ and CD8⁺ markers to identify T cell subpopulations. The relative gated percent of CD3⁺CD4⁺ T cells (left bar in each pair) shows that cART cotherapy with FX101 (FX+cART) comparatively prevents loss of CD4⁺ T cells versus FX101 alone (FX), further benefiting animals achieving a period of virus suppression (“Sup”, <50 RNA copies/mL). The black line depicts the CD4⁺:CD8⁺ T cell ratios, with values noted. The CD4⁺:CD8⁺ ratios are highest for animals achieving a period of suppression (Sup).

FIG. 22 illustrates flow cytometric analysis of whole blood that was collected at 25 Weeks post infection (10 weeks no treatment, 15 weeks of drug treatment) from six animals in the second Cohort, including two animals in each treatment group except for the control group (which were part of Cohort 1). One hundred microliters of whole blood were labeled using fluorescent antibodies for CD3⁺, CD4⁺ and CD8⁺ markers to identify T cell subpopulations. The relative gated percent of CD3⁺CD4⁺ T cells (left bar in each pair) shows that cART cotherapy with FX101 (FX+cART) comparatively prevents loss of CD4⁺ T cells versus FX101 alone (FX), further benefiting animals achieving a period of virus suppression (“Sup”, <50 RNA copies/mL). The black line depicts the CD4⁺:CD8⁺ T cell ratios, with values noted.

FIG. 23 illustrates flow cytometric analysis of whole blood that was collected at 27 Weeks post infection (10 weeks no treatment, 15 weeks of drug treatment followed by two weeks off cART therapy) from six animals in the second Cohort, including two animals in each treatment group except for the control group. One hundred microliters of whole blood were labeled using fluorescent antibodies for CD3⁺, CD4⁺ and CD8⁺ T cell markers to identify T cell subpopulations. The relative gated percent of CD3⁺CD4⁺ T cells (left bar in each pair) shows that cART cotherapy with FX101 (FX+cART) comparatively prevents loss of CD4+ T cells versus FX101 alone (FX), further benefiting animals achieving a period of virus suppression (“Sup”, <50 RNA copies/mL). The black line depicts the CD4⁺:CD8⁺ ratios, with values noted. Animals had been off cART therapy for only 2 weeks, with the cotherapeutically suppressed (Sup) FX+cART animal comparatively preserving loss of CD4⁺ T cells and exhibiting a correspondingly lower CD8⁺ T cell response as compared with other cART-treated animals. Additionally, the proportion of CD3⁺CD8⁺ T cells (right bar in each pair) is correspondingly lower with cotherapy. The line with labeled values (read from right axis) represents the ratio of CD4⁺/CD8⁺ T cells (CD4:CD8), which is greater than 1.0 for animals achieving virus suppression during therapy and for some of these animals post treatment cessation, and clinically beneficial.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B. A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” can refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

In addition, the steps of the methods described herein can be performed in any suitable order, including simultaneously.

The term “about” or “approximately” can refer to within an acceptable error range for the particular value as determined by one of ordinary skill in the art, which will depend in part on how the value is measured or determined, i.e., the limitations of the measurement system. For example, “about” can refer to within 3 or more than 3 standard deviations, per the practice in the art. Alternatively, “about” can refer to a range of up to 20%, e.g., up to 10%, up to 5%, or up to 1% of a given value. Alternatively, particularly with respect to biological systems or processes, the term can refer to within an order of magnitude, e.g., within 5-fold, or within 2-fold, of a value.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The “median” value can refer to the median value obtained from a collection of measures or a population of subjects. The median values can be previously determined reference values or can be contemporaneously determined values.

It will be understood that, although the terms first, second, etc. can be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Unless defined otherwise, all technical and scientific terms used herein have the meaning commonly understood by a person skilled in the art to which this invention belongs. The following references provide one of ordinary skill in the art with a general definition of many of the terms used herein: Singleton et al, Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991): Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984): “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al, ed., 1994); “A Practical Guide to Molecular Cloning” (Perbal Bernard V., 1988); “Phage Display: A Laboratory Manual” (Barbas et al, 2001). The contents of these references and other references containing standard protocols, widely known to and relied upon by those of skill in the art, including manufacturers' instructions are hereby incorporated by reference as part of the presently disclosed subject matter.

As described herein, aspects of the invention are drawn towards compositions and methods for treating, preventing, alleviating a symptom of, suppressing, providing remission from the requirement of continuous therapeutics, or curing a viral infection. In an embodiment, the method comprises administering to a subject a compound according to Formula (1a) or Formula (1b)

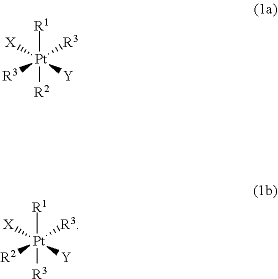

wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻; wherein R¹ is NO₂. COOH, COOR², OR, COR, OH or SO₃H; wherein R² is F, Cl, Br, I, CN, SCN, NCS, NO₃, ONO, OHSO₃, OH₂PO₃, OHSO₃, SO₃H, OH, OR², OS(CH₃)₂, OCOR², OCOOR², OSO₂CH₃, OS(CH₃, SH, SR², SC(NH₂)₂, S₂CN(R)₂, OSiO₃, OSO₂H, OHSeO₂, NHCOH, NH₂CHO, NH₂CH₂OH, NH₂C(OH)₃, NH₂CH(OH)₂, NHCOR², carboxylate (CO₂R²) sulfate (SO₄)₂, phosphate (HPO₄)²⁻, selenate (SeO₄)²⁻, or silicate (SiO₄)², an alkyl, alkoxy, cycloalkyl, cycloalkoxy, aryl, aryloxy, alky carbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH—COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

The term “treating”, “to treat”, or “treatment” can refer to an approach for obtaining beneficial or desired results. For example, a beneficial or desired result can include, but is not not limited to, alleviation of a symptom and/or diminishment of the extent of a symptom and/or preventing a worsening of a symptom associated with a disease or condition. In one embodiment, “treatment” or “treating” can include one or more of the following: a) inhibiting the disease or condition (for example, decreasing one or more symptoms resulting from the disease or condition, and/or diminishing the extent of the disease or condition); b) slowing or arresting the development of one or more symptoms associated with the disease or condition (for example, stabilizing the disease or condition, delaying the worsening or progression of the disease or condition); and/or c) relieving the disease or condition, for example, causing the regression of clinical symptoms, ameliorating the disease state, delaying the progression of the disease, increasing the quality of life, and/or prolonging survival.

The term “prevent,” “prevention,” or “preventing” can refer to any method to partially or completely prevent or delay the onset of one or more symptoms or features of a disease, disorder, and/or condition. Prevention can be administered to a subject who does not exhibit signs of a disease, disorder, and/or condition.

The phrase “suppressing a viral infection” can refer to suppressing any aspect of viral infection, such as viral replication, time course of infection, amount (titer) of virus, lesions, and/or one or more symptoms is curtailed, inhibited, or reduced (in terms of severity and/or duration) in a subject with a compound of Formula (1a) and/or (1b), optionally, together with at least one additional antiviral therapy, as compared to an aspect of viral infection in an individual or a population of individuals not treated in accordance with the invention. Reduction in viral load includes, but is not limited to, elimination of the virus from an infected site, from a tissue, or from an individual. Viral infection can be assessed by any means known in the art, including, but not limited to, measurement of virus particles, viral nucleic acid or viral antigens, detection of symptoms and detection and/or measurement of anti-virus antibodies. Anti-virus antibodies are widely used to detect and monitor viral infection and generally are commercially available.

“Providing remission from the requirement of continuous therapeutics” can refer to reducing the dosing frequency or the extent and/or time course requirement of another therapeutic required to control an infection in a subject treated with a compound of Formula (1a) and/or (1b), optionally, together with at least one additional antiviral therapy.

The term “curing” a viral infection can refer to eliminating the detection of a virus from accessible tissues in a subject previously infected with a virus, below the clinical limits for detecting that virus using methods known in the art, including those commercially available.

As used herein, “delaying” development of a viral infection or a symptom of viral infection can refer to defer, hinder, slow, retard, stabilize, and/or postpone development of the disease or symptom when compared to not using the method(s) of the invention. This delay can be of varying lengths of time, depending on the history of the disease and/or individual being treated. As is evident to one skilled in the art, a sufficient or significant delay can, in effect, encompass prevention, in that the individual does not develop the disease.

“Reducing severity of a symptom” or “ameliorating a symptom” of viral infection can refer to a lessening or improvement of one or more symptoms of viral infection as compared to not administering a compound of Formula (1a) and/or (1b), optionally, together with at least one additional antiviral therapy. “Reducing severity” can also refer to shortening or reduction in duration of a symptom. These symptoms are known to the skilled artisan and include, but are not limited to reducing the viral setpoint, reducing the viral load in plasma, cerebrospinal fluid or in an anatomical site, reducing the proportion of cells harboring proviral DNA, reducing the loss of CD4⁺ T cells, improving innate or adaptive immunologic functions, reducing inflammation, supporting a period of drug-free viral control, reducing the transmission of virus, reducing comorbidities associated with an infection, or reducing damage to organs or tissues.

“Preventing a symptom of infection” can refer to that the symptom(s) does not appear after exposure to the virus. Examples of symptoms have been described herein.

“Reducing duration of viral infection” can refer to the length of time of viral infection (usually indicated by symptoms) is reduced, or shortened, as compared to not administering a compound of Formula (1a) and/or (1b), optionally, together with at least one additional antiviral therapy.

The term “infected subject” can refer to an individual who has been infected by a virus, such as a virus described herein. Symptoms of such virus infections are well known in the art and have been described herein.

The effectiveness of treating, preventing, alleviating a symptom of, suppressing, providing remission from the requirement of continuous therapeutics, or curing a viral infection can be indicated by methods known to the skilled artisan, for example, measuring viral loads. Measuring viral loads, for example, can comprise reductions in frequencies of virus-producing cells, reductions in viral load measured from cerebrospinal fluid, reductions in viral load measured from blood, increased IL-21 productions, the development of broadly neutralizing antibodies, a reduction in viral setpoint, a reduction in the frequency of provirus in peripheral blood mononuclear cells, a reduction in the frequency of cell-associated viral RNA, a reduction in the frequency of cell-associated viral DNA, measuring cytokines, chemokines or transcription of viral proteins, or any combination thereof. For example, the effectiveness of treating, preventing, alleviating a symptom of, suppressing, providing remission from the requirement of continuous therapeutics, or curing a viral infection can be indicated from blood, lymph or tissue, or using tissue aspirations, measuring neutralizing antibody titers, measuring viremia, sequencing or identifying RNA or DNA, performing flow cytometric analyses, measuring provirus in host cells (for example, PBMCs), measuring viral DNA, measuring viral RNA and/or measuring viral proteins using methods known iin the art.

Embodiments herein can be used to reduce virus replication or viral reservoirs in tissues. Embodiments herein can be used to limit viral protein-host protein and viral protein-nucleic acid interactions, including those that interfere with host immune responses to virus infection. Embodiments herein can be used to support the development of autologous broadly neutralizing antibodies against virus infections. Embodiments herein can be used to increase the frequency of interleukin-21-producing cells in lymph nodes. Embodiments herein can be used to reduce the frequency of virus or provirus in host cells.

As described herein, a viral infection can be treated, prevented, alleviated, or cured by administering a compound according to Formula (1a) or Formula (1b).

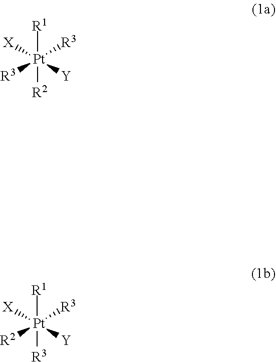

In embodiments, the platinum (Pt) compounds of the invention can be used because of their octahedral (also known as trigonal bipyramidal) coordination geometries in the Pt(IV) oxidation state, and square planar coordination geometries in the Pt(II) oxidation state. Most metals, including zinc, coordinate in tetrahedral geometries. Hence, the coordination of zinc by four protein residues defines the protein tertiary structure orientation for the functionality of that protein. When Pt compounds displace the zinc atom and coordinate to that protein, the protein tertiary structure is altered, changing the functionality of the associated protein. This is particularly important to viral proteins coordinating zinc because these proteins are sometimes polyproteins being cleaved stepwise with distinct functions associated with each cleavage, and because the diverse functions of those proteins involve numerous interactions between viral and host proteins and nucleic acids. Thus, the zinc binding domain has been identified as critical for multiple viral activities, including manipulation of host cell proteins. Without wishing to be bound by theory, the unique octahedral and planar coordination geometries of Pt compounds preferentially distinguish this metal therapeutic for disrupting zinc-containing viral proteins. Other metals with octahedral or square planar coordination geometries useful for disrupting the zinc finger domains of multiple viruses including, but not limited to, complexes of Palladium (Pd), Rhodium (Rh), Rubidium (Ru), Iridium (Ir), and Gold (Au). Preferred oxidation states of the preceding metal complexes include Ir(I), Ir(III), Rh(I), Rh(III), Au(III), Ru(II), Ru(III) and Pd(II).

In embodiments, X and Y can be, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻.

In embodiments, R¹ can be NO₂, COOH, COOR², OR, COR, OH or SO₃H.

In embodiments, R² can be F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl.

In embodiments, R³ can be NH₃, NH₂R², NH(R²)₂, N(R²)₃, NH₂COR². NH₂COH, NH₂CHO, NH₂CH₂OH, NH₂C(OH)₃, NH₂CH(OH), NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative.

In embodiments, the orientation of X and Y are trans to one another. Platinum compounds of the cis configuration such as cisplatin, with adjacent chlorides, can crosslink DNA as alkylating agents, and are useful chemotherapeutics for the treatment of various cancers. Trans geometries of platinum compounds such as transplatin, have not been therapeutically effective for treating cancer, as the trans chloride geometries form fewer and different adducts with DNA than do cisplatin analogues. Pt(IV) prodrugs are largely intended to overcome systemic toxicities and become reduced to their corresponding Pt(II) cis-configuration geometries as cancer drugs. Pt(IV) oxidation states are preferred. Trans geometries of X and Y are generally preferred over cis geometries. Coordination ligands that are oriented approximately 180° (trans) around the central atom versus 90° (cis) result in greater perturbations to the approximately 109° (tetrahedral) coordination of the zinc atom and have demonstrated comparatively better activities in vitro.

Embodiments can further comprise a pharmaceutically acceptable salt, crystal, co-crystal, prodrug, or solvate thereof, or any combination thereof.

“Pharmaceutically acceptable salt” can refer to a physiologically or toxicologically tolerable salt, including but not limited to, when appropriate, pharmaceutically acceptable base addition salts and pharmaceutically acceptable acid addition salts. For example (i) where a compound contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see, for example, “Handbook of Pharmaceutical Salts: Properties, Selection and Use” by Stahl and Wermuth (Wiley-VCH, Weinheim, Germany, 2002).

The term “prodrug” can refer to a compound which is convertible in vivo by metabolic means (e.g. by hydrolysis, reduction or oxidation) to a compound of the invention. Suitable groups for forming prodrugs are described in ‘The Practice of Medicinal Chemistry, 2nd Ed. pp 561-585 (2003) and in F. J. Leinweber, Drug Metab. Res., 1987, 18, 379.

The compounds of Formula (1a) and/or (1b) can exist in both unsolvated and solvated forms. The term ‘solvate’ can refer to a molecular complex comprising the compound of the invention and a stoichiometric amount of one or more pharmaceutically acceptable solvent molecules, for example, ethanol. The term ‘hydrate’ can be employed when the solvent is water.

The compounds of Formula (1a) and/or (1b) can exist in any of the regulatory classifications of active pharmaceutical ingredient crystal or co-crystal solid-state forms. The crystal or co-crystal form can be considered in the same way as would any other morphic form such as a polymorph, solvate or hydrate. Co-crystals can be composed of two or more different molecules, typically an active pharmaceutical ingredient and an excipient, wherein the co-crystal can exhibit various physical properties affecting stability, bioavailability, or pharmacokinetics.

Where compounds used in the compositions of the invention exist in one or more geometrical, optical, enantiomeric, diastereomeric and tautomeric forms, including but not limited to cis- and trans-forms, E- and Z-forms, R-, S- and meso-forms, keto-, and enol-forms, then, unless otherwise stated, a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

As one example, embodiments are drawn towards compositions and methods for treating, preventing, alleviating a symptom of, suppressing, providing remission from the requirement of continuous therapeutics, or curing a viral infection. In an embodiment, the method comprises administering to a subject a compound according to Formula (1a) or Formula (1b),

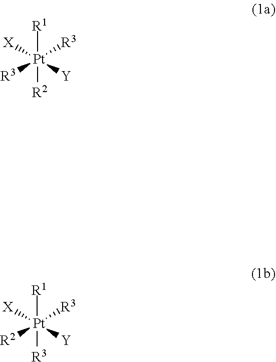

wherein X and Y are Cl, R¹ is NO₂, R² is Cl, and R³ is NH₃.

Non-limiting examples of other compounds that can utilized as described herein comprise administering to a subject a compound according to Formula (1a) or Formula (1b),

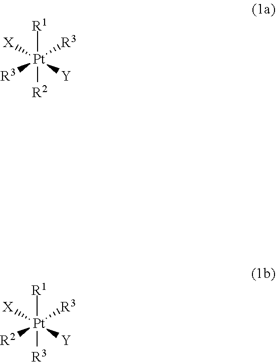

wherein X and Y are independently, nucleophilic exchangeable leaving groups, R¹ is NO₂ or OH.

R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)₂, phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OSO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl.

R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

Non-limiting examples of other compounds that can be utilized as described herein comprise those described in U.S. Pat. Nos. 8,026,382; 8,425,921; 8,895,610 and 9,132,115, each of which are incorporated by reference herein in their entireties.

Therapeutic application of compounds and/or compositions described herein can be accomplished by any suitable therapeutic method and technique presently or prospectively known to those skilled in the art. For example, compounds of Formula (1a) and/or (1b), optionally together with at least one additional antiviral therapy, can be administered by any suitable route known in the art including, for example, oral, nasal (e.g., via aerosol inhalant), rectal, ex vivo (reintroduction of treated tissues), and parenteral routes of administration. As used herein, the term parenteral includes topical, subdermal (e.g., as in an implant), subcutaneous, intradermal, intravenous, intramuscular, intraperitoneal, and intrasternal administration, such as by injection. Administration of the subject platinum compounds of the invention can be continuous or at distinct intervals as can be readily determined by a person skilled in the art.

Compounds of Formula (1a) and/or (1b), optionally together with at least one additional antiviral therapy, can be administered utilizing liposome technology, antibody-conjugation, peptide-conjugation, nanotechnology (such as carbon nanotubes, gold nanospheres, or nanoslow-release capsules), polymeric sugars, electroporation, implantable pumps, and biodegradable containers. Certain of these delivery methods can, advantageously, provide a uniform dosage over an extended period of time while others provide immediate and/or local targeting. The platinum compounds of the present invention can also be administered in their salt derivative forms or crystalline forms known to those of ordinary skill in the art.

Compounds of Formula (1a) and/or (1b), optionally together with at least one additional antiviral therapy, can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. For example, the compositions of the subject invention can be formulated such that a bioeffective amount of the platinum compound is combined with a suitable carrier in order to facilitate effective administration of the composition. The compounds and/or compositions used in the present methods can also be in a variety of forms. These include, for example, solid, semi-solid, and liquid dosage forms, such as tablets, pills, powders, liquid solutions or suspension, suppositories, injectable and infusible solutions, aerosol particle, and sprays. The preferred form depends on the intended mode of administration and therapeutic application. The compositions can also include conventional pharmaceutically acceptable carriers and diluents which are known to those skilled in the art. Examples of carriers or diluents for use with the subject platinum compounds include ethanol, ethyl acetate, dimethyl sulfoxide, glycerol, alumina, starch, and equivalent carriers and diluents. To provide for the administration of such dosages for the desired therapeutic treatment, pharmaceutical compositions of the invention will advantageously comprise between about 0.1% and 99%, and especially, 1 and 15% by weight of the total of one or more of the subject platinum compounds based on the weight of the total composition including carrier or diluent.

“In combination with or conjunction with” can refer to administration of a compound of Formula (1a) or Formula (1b), or a pharmaceutically acceptable salt, crystal or co-crystal thereof, together with at least one other antiviral therapy. Administration of a compound of Formula (1a) or Formula (1b), or a pharmaceutically acceptable salt, crystal or co-crystal thereof, together with at least one other antiviral therapy can refer to and include “simultaneous administration” or “sequential administration”.

“Simultaneous administration” can refer to administration of a compound of Formula (1a) and/or Formula (1b), or a pharmaceutically acceptable salt, crystal or co-crystal thereof, and at least one additional antiviral therapy to a subject in need of treatment, when such components are formulated together into a single dosage form which releases said components at substantially the same time to said patient. “Simultaneous administration” can also refer to administration of a compound of Formula (1a) or Formula (1b), or a pharmaceutically acceptable salt, crystal or co-crystal thereof, and at least one additional antiviral therapy to a subject in need of treatment, when such components are formulated apart from each other into separate dosage forms which are taken at substantially the same time by said patient, whereupon said components are released at substantially the same time to said patient.

“Sequential administration” can refer to administration of a compound of Formula (1a) or Formula (1b), or a pharmaceutically acceptable salt, crystal or co-crystal thereof, and at least one additional antiviral therapy to a subject in need of treatment, when such components are formulated apart from each other into separate dosage forms which are taken at consecutive times by said patient with a significant time interval between each administration, whereupon said components are released at substantially different times to said patient.

A compound of Formula (1a) or Formula (1b), or a pharmaceutically acceptable salt, crystal or co-crystal thereof, and at least one additional antiviral therapy to a subject in need of treatment, can be formulated together into a single dosage form which releases said components in a controlled manner whereupon they are concurrently, consecutively, and/or alternately administered at the same and/or different times by said patient. In embodiments, each part can be administered by either the same or different route.

In embodiments, a therapeutically effective amount of a compound according to Formula (1a) or Formula (1b), optionally together with a therapeutically effective amount of at least one additional antiviral agent, can be administered to a subject. The term “therapeutically effective amount” can refer to the amount of an agent required to provide a meaningful patient gain. In general, the ultimate goal of antiviral treatment is the suppression of viral load, improved immunologic function, prevention of the loss of CD4⁺ T cells, reduction of viral reservoirs, development of autologous broadly neutralizing antibodies, reduction of inflammation, improved quality of life and reduced morbidity and mortality.

The terms “individual”, “patient” and “subject” can be used interchangeably. They can refer to a mammal (e.g., a human) which is the object of treatment, or observation. Typical subjects to which compositions and methods described herein can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

As described herein, aspects of the invention are drawn towards compositions and methods for treating, preventing, alleviating a symptom of, suppressing, providing remission from the requirement of continuous therapeutics, or curing a viral infection.

A viral infection can refer to both an infection with a virus (e.g., HIV, SIV, SHIV, FIV, and the like) and a disease caused by an infection with a virus.

In embodiments, the virus comprises a lentivirus. A“lentivirus” can refer to a genus of retroviruses that can infect dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in nonhuman primates.

Non-limiting examples of lentiviruses that can be treated, prevented, alleviated or cured comprises HIV, SIV, SHIV, or FIV.

“HIV infection”, “ARC”, “AIDS” and related terms are used as understood by physicians in the field of HIV infection. As used herein, “HIV” or “Human Immunodeficiency Virus” can refer to HIV-1 and/or to HIV-2.

“ARC” can refer to symptomatic pathologies in HIV infection that include low grade fever, unexplained weight loss, diarrhea, opportunistic infections, and generalized lymphadenopathy.

“AIDS” can refer to acquired immunodeficiency syndrome and is defined in HIV infection with either a CD4⁺ T cell count below 200 cells per μL or the occurrence of specific diseases associated with HIV infection, including the development of opportunistic infections, including but not limited to, pneumocystis pneumonia, cachexia (wasting syndrome), esophageal candidiasis and recurrent respiratory tract infections.

In embodiments, the virus is not HIV, SIV, SHIV, or FIV.

In embodiments, the virus that can be treated, prevented, alleviated or cured comprise viruses of the Retroviridae, Coronaviridae, Paramyxoviridae, Togaviridae, Flaviviridae, Bunyavirales or Hepadnavirus orders/families. Each of these comprises viruses expressing viral proteins containing zinc binding domains that can be disrupted by a therapeutically effective amount of compounds as described herein (e.g., Formula (1a) and/or (1b)), thus altering the activities of the associated viral proteins. Activities of these viral proteins include not only those associated with virus replication and integration, but may also involve disease severity or host immune responses through viral protein-host protein and viral protein-nucleic acid interactions.

Non-limiting examples of such viruses comprise an Oncoretrovirus, Human T-lymphotropic virus (HTLV), Feline lymphotropic virus (FeLV), Spumavirus, a Nidovirus, Severe Respiratory Syndrome Corona Virus 2 (SARS-CoV-2), Middle East Respiratory Syndrome (MERS-CoV), Severe Respiratory Syndrome Corona Virus 1 (SARS-CoV-1), a Henipavirus, Measles virus (MeV), Nipah virus (NiV), Mumps virus (MuV), Sendai virus (SeV), Parainfluenza virus 5 (PIV-5), Human parainfluenza virus (HPIV); Hendra virus (HeV), Newcastle Disease virus (NDV), an Alphavirus, Chikungunya virus (CHIKV), Sindbis virus (SINV), Dengue virus-1, Dengue virus-2, Dengue virus-3, Dengue Virus-4, West Nile virus (WNV), Japanese encephalitis virus (JEV), Zika virus, Yellow Fever Virus (YFV), a Nairovirus, Crimean Congo Hemorrhagic Fever (CCHF) virus, a Hantavirus, an Orthobunyavirus, an Arenavirus, or Hepatitis B virus (HBV).

In embodiments, a compound of Formula (1a) and/or (1b) can be administered monotherapeutically, or optionally, in combination with at least one additional antiviral therapy. The term “antiviral therapy” can refer to a small molecule, biological molecule, antibody or immunotherapy (e.g., immune-based therapy, immune checkpoint inhibitor, chimeric antigen receptor T cell/natural killer cell, or other cell therapy engineered to treat a virus infection), vaccine, or the removal of infected cells. “Antiviral therapy” is intended to limit or reduce the multiplication of virus in the body, reduce the viral load, relieve symptoms, prevent severe illness and/or prevent death. In embodiments, the at least one additional antiviral therapy can comprise standard of treatment care.

In embodiments, the subject has previously been treated with at least one additional antiviral therapy that has not eliminated the virus from the subject. Non-limiting examples of an antiviral therapy can comprise a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

For example, the antiviral therapy can comprise abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1, nirmatrelvir, ritonavir, remdesivir, molnupiravir, ribavirin, T-705, EIDD-1931, NITD008, Infergen®, enviroxime, pirodavir, acyclovir, adefovir, amantadine, ampligen, cidofovir, daclatasivir, ensitrelvir, foscamet, ganciclovir, letermovir, methisazone, moroxydine, nitazosanide, oseltamivir, peramivir, pleconaril, rimantadine, simeprevir, sofosbuvir, taribavirin, telaprevir, telbibudine, tromantadine, umifenovir, valaciclovir, valganciclovir, vidarabine, zanamivir, or any combination thereof.

In embodiments, the antiviral therapy can comprise a broadly neutralizing antibody. A “broadly neutralizing antibody” can refer to neutralizing antibodies which neutralize multiple virus strains by targeting more highly conserved epitopes of the virus. Tier 2 and Tier 3 antibodies are broadly neutralizing antibodies; Tier 1 antibodies are not broadly neutralizing. Non-limiting examples of the broadly neutralizing antibody can comprise VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

A “retrovirus” can refer to a virus having an RNA genome that inserts a DNA copy of its genome into the DNA of a host cell it infects. This integrated viral genome is referred to as “provirus” or “proviral DNA”. In embodiments, the antiviral therapy can be an antiretroviral therapy.

In embodiments, the antiviral therapy comprises a combination antiretroviral therapy (cART). The phrase “combination antiretroviral therapy” can refer to a treatment that uses a combination of drugs, such as a combination of two or more drugs, to treat a viral infection, such as an HIV infection. Non-limiting examples of cARTs comprise ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir, emtricitabine, and tenofovir alafenamide); darunavir, tenofovir alafenamide hemifumarate, emtricitabine, and cobicistat; efavirenz, lamivudine, and tenofovir disoproxil funarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride: cabotegravir and rilpivirine; cabotegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine; lamivudine+abacavir+zidovudine; lamivudine+abacavir; lamivudine+tenofovir disoproxil funarate; lamivudine+zidovudine+nevirapine; lopinavir+ritonavir; lopinavir+ritonavir+abacavir+lamivudine; lopinavir+ritonavir+zidovudine+lamivudine; tenofovir+lamivudine; and tenofovir disoproxil funarate+emtricitabine+rilpivirine hydrochloride; lopinavir, ritonavir, zidovudine and lamivudine; Vacc-4x and romidepsin; and APH-0812.

Aspects of the invention are also drawn towards methods of treating, preventing, alleviating a symptom of, suppressing, providing remission from the requirement of continuous therapeutics, or curing a viral infection, or alleviating a symptom thereof by administering to a subject a first agent and a second agent, wherein the first agent comprises an antiviral therapy, and wherein the second agent comprises a compound according to Formula (1a) or Formula (1b)

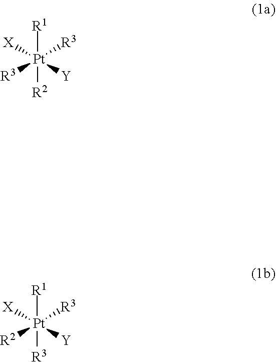

wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻; wherein R¹ is NO₂, COOH, COOR², OR, COR, OH or SO₃H; wherein R² is F, Cl, Br, I, CN, SCN, NCS, NO₂. ONO, OHSO₃, OH₂PO₃, OHSO₂, SO₃H, OH, OR², OS(CH₃)₂, OCOR², OCOOR², OSO₂CH₃, OS(CH)₂, SH, SR², SC(NH₂)₂, S₂CN(R²)₂, OSiO₂, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl; wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

Aspects of the invention are also drawn towards methods of treating, preventing, alleviating a symptom of, suppressing, providing remission from the requirement of continuous therapeutics, or curing a viral infection, or alleviating a symptom thereof, wherein the first agent and the second agent are administered simultaneously or sequentially.

Aspects of the invention are also drawn towards methods of treating, preventing, alleviating a symptom of, suppressing, providing remission from the requirement of continuous therapeutics, or curing a viral infection, or alleviating a symptom thereof, wherein the subject is infected with more one or more viruses. Nonlimiting examples include coinfections of HIV with hepatitis B, hepatitis C, or SARS-Cov-2, that can be treated using the methods of this invention.

For example, embodiments comprise the use of a therapeutically effective amount of a compound of Formula (1a) or Formula (1b), or a pharmaceutically acceptable salt, crystal or co-crystal thereof, alone or in combination with or in conjunction with a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, a capsid inhibitor; a combination therapy (e.g., cART), a latency reversing agent, an immune-based therapy, a chimeric antigen receptor therapy, a PI3K inhibitor, an antibody, a bispecific antibody, or antibody-like protein, an antiviral vaccine, and/or an immune-checkpoint inhibitor.

For example, one aspect of the present invention is the use of a therapeutically effective amount of a compound of Formula (1a) or Formula (1b), or a pharmaceutically acceptable salt, crystal or co-crystal thereof, alone or in combination with or in conjunction with a latency reversing agent, an immune-based therapy, a PI3K inhibitor, an antibody, a bispecific antibody, or an “antibody-like” therapeutic protein, or combinations thereof.

Another aspect of the present invention is the use of a therapeutically effective amount of a compound of Formula (1a) or Formula (b), or a pharmaceutically acceptable salt, crystal or co-crystal thereof, alone or in combination with or in conjunction with a broadly neutralizing antibody. The broadly neutralizing antibody may be selected from the group comprising VRC01, VRC07, 3BNC117, 10-1074, PGDM1400, 10E8, N6, 4/iMab, PGT121, elipovimab, N6LS, PGT-121, Teropavimab (GS-5423), Zinlirvimab (GS-2872) and Elipovimab (GS-9722).

One aspect of the present invention is the use of a therapeutically effective amount of a compound of Formula (1a) or Formula (1b), or a pharmaceutically acceptable salt, crystal or co-crystal thereof, together with at least one other antiviral therapeutic agent selected from the group consisting of a nucleoside/nucleotide reverse transcriptase inhibitor (RTI) selected from the group consisting of Abacavir (ABC), Didanosine (ddI), emtricitabine (FTC), lamivudine (3TC), stavudine (d4T), tenofovir (TFV, TFV-DP, TDF or TAF), zalcitabine (ddC), and zidovudine (AZT), or a pharmaceutically acceptable salt, crystal, co-crystal or solvate thereof; a non-nucleoside/nucleotide reverse transcriptase inhibitor (NNRTI) selected from the group consisting of delavirdine (DLV), doravirine (DOR), efavirenz (EFV), etravirine (ETR), nevirapine (NVP), and rilpivirine (RPV), MK-8507, elsulfavirine (VM1500) or a pharmaceutically acceptable salt, crystal, co-crystal or solvate thereof; a protease inhibitor (PI) selected from the group consisting of atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), and tipranavir (TPV), or a pharmaceutically acceptable salt, crystal, co-crystal or solvate thereof. PI-based regimens using pharmacokinetic (PK) enhancement with either cobicistat (COBI) or RTV (also called PK boosting) increase concentration and prolong the half-lives of the PI; an integrase strand transfer inhibitor (INSTI) selected from the group consisting of bictegravir (BIC), dolutegravir (DTG), elvitegravir (EVG), raltegravir (RAL) and cabotegravir (CAB), or a pharmaceutically acceptable salt, crystal, co-crystal or solvate thereof; a fusion inhibitor selected from the group consisting of enfuvirtide (T-20), T-1249, albuvirtide, BMS-986197, enfuvirtide biobetter, enfuvirtide biosimilar. HIV-1 fusion inhibitors (P26-Bapc), ITV-1, ITV-2, ITV-3, ITV-4, PIE-12 trimer, and sifuvirtideor a pharmaceutically acceptable salt, crystal, co-crystal or solvate thereof; an entry inhibitor selected from the group consisting of a CCR5 inhibitor, leronlimab (PRO-140), Sch-C, Sch-D, TAK-220, PRO-140, UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral envelope adhesion inhibitor, a viral gp120 inhibitor, a viral gp160 inhibitor, or a viral gp41 inhibitor, or a pharmaceutically acceptable salt thereof; a virus budding or maturation inhibitor (MI) selected from the group consisting of bevirimat (BVM), GSK3640254 (GSK254), GSK3739937 or a pharmaceutically acceptable salt thereof; and/or a capsid inhibitor selected from the group consisting of lenacapavir (LCV), PF74, GS-CA1, or a pharmaceutically acceptable salt or solvate thereof.

One aspect of the present invention is the use of a therapeutically effective amount of a compound of Formula (1a) or Formula (1b), or a pharmaceutically acceptable salt, crystal, co-crystal thereof, together with at least one other antiviral therapy. Non-limiting examples of other antiviral therapies comprise acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

Examples of latency reversing agents for treating HIV infection include histone deacetylase (HDAC) inhibitors, proteasome inhibitors such as velcade, protein kinase C (PKC) activators, BET-bromodomain 4 (BRD4) inhibitors, ingenol, vorinostat, (suberanilohydroxamic acid), Interleukin-15 (IL-15), JQ1, disulfram, amphotericin B, and ubiquitin inhibitors such as largazole analogs, and GSK-343.

Examples of HDAC inhibitors include romidepsin, vorinostat, belinostat and panobinostat.

Examples of PKC activators include indolactam, prostratin, bryostatin, DPP, ingenol B, phorbol esters and Diacylglycerol (DAG)-lactones.

Examples of Immune-Based Therapies include toll-like receptors modulators such as TLR1, TLR2, TLR3, TLR4, TLR5, TLR6, TLR7, TLR8, TLR9, TLR10, TLR11, TLR12, and TLR13; programmed cell death protein 1 (PD-1) modulators; programmed death-ligand 1 (PD-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea: mycophenolate mofetil (MPA) and its ester derivative mycophenolate mofetil (MMF); ribavirin; vesatolimod; R848; AZD8848; imiquimod, TMX-202, TMX-302, TMX-306, resiquimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103.

Examples of Phosphatidylinositol 3-Kinase (PI3K) Inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

Examples of HIV Antibodies, Bispecific Antibodies, and “Antibody-Like” Therapeutic Proteins include DARTs®, DUOBODIES®, BITESS, XmAbs®, TandAbs®, Fab derivatives, bnABs (broadly neutralizing HIV-1 antibodies), BMS-936559, TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo and MB-66.

Examples of other antibodies for treating HIV include bavituximab, UB-421, C2F5, C2G12, C4E10, C2F5+C2G12+C4E10, 3-BNC-117, PGT145, PGT121, MDXO10 (ipilimumab), VRC01, A32, 7B2, 10E8, VRC-07-523, VRC-HIVMAB080-00-AB, MGD-014 and VRC07.

Examples of HIV Vaccines include peptide vaccines, recombinant subunit protein vaccines, live vector vaccines, DNA vaccines, CD4-derived peptide vaccines, vaccine combinations, rgp120 (AIDSVAX), ALVAC HTV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-05, VAC-3 S, multiclade DNA recombinant adenovirus-5 (rAd5), Pennvax-G, Pennvax-GP, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, AGS-004, AVX-101, AVX-201, PEP-6409, SAV-001, ZhV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, and rAd5 gag-pol env A/B/C vaccine.

Examples of antiviral checkpoint inhibitors include PD-1 inhibitors (programmed death cell protein-1); CTLA-4 inhibitors (anti-cytotoxic T-lymphocyte-associated protein 4); TIM-3 inhibitors (T cell immunoglobulin and mucin domain-containing protein 3); TIGIT inhibitors (T cell immunoreceptor with Ig and ITIM domains); and LAG-3 inhibitors (lymphocyte-activation gene 3).

It will be appreciated by one of skill in the art that therapeutic agents listed above may be included in more than one of the classes listed above. The particular classes are not intended to limit the functionality of the compounds listed in these classes.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to reduce the viral load as compared with the standard of care alone.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to reduce persistent sources of virus in anatomical reservoirs, including but not limited to blood, lymphatic tissues, spleen and/or the central nervous system.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to reduce the plasma viremia setpoint.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to reduce the frequency of provirus in target host cells, including but not limited to peripheral blood mononuclear cells or those residing in compartmental reservoirs such as lymphatic tissues, spleen or the central nervous system.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to support the development of broadly neutralizing antibodies.

For example, a therapeutically effective amount of a compound of Formula (1a) or Formula (1b), or a pharmaceutically acceptable salt, crystal or co-crystal thereof, can be used alone or in combination with or in conjunction with at least one other antiviral therapy to increase IL-21 expression in lymph nodes, reduce inflammatory protein-10 (IP-10; CXCL10) in cerebrospinal fluid, reduce macrophage inflammatory protein-1-beta (MIP-1b), reduce monocyte chemoattractant protein-1 (MCP-1), reduce Stromal Cell-Derived Factor-1 (SDF-1; CXCL12) or ITAC (CXCL11).

Administration of a compound of Formula (1a) or Formula (1b), or a pharmaceutically acceptable salt, crystal or co-crystal thereof, is active in combination with or in conjunction with a wide variety of other antiviral agents and may be particularly useful toward long term remission or curative therapeutic approaches for treating virus infections. In embodiments, administration of a compound of Formula (1a) or Formula (1b), or a pharmaceutically acceptable salt, crystal or co-crystal thereof, exhibits synergistic antiviral activity when used in combination with or in conjunction with a variety of other antiviral agents. The term “synergistic” can refer to a greater effect and/or longer suppressive duration when two or more drugs are used in combination than would be expected from adding the individual effects of the two components or example greater than two times, greater than three times, greater than five times or greater than ten times what would be expected from adding the individual effects of the two components.

In embodiments, a compound of Formula (1a) or Formula (1b), or a pharmaceutically acceptable salt, crystal or co-crystal thereof, can be administered as a pharmaceutical composition, and the active ingredient of the composition comprises a compound of Formula (1a) or Formula (1b), or a pharmaceutically acceptable salt, crystal or co-crystal thereof, either alone or in combination with or in conjunction with at least one other antiviral agent used to treat a virus infection.

The pharmaceutical composition can be manufactured with a pharmaceutically acceptable carrier or vehicle and may include conventional excipients. “Excipient” can refer to a substance formulated together with the active pharmaceutical ingredient(s) of a medication, included for the purpose of long-term stabilization, bulking up formulations that contain potent active pharmaceutical ingredients in measured amounts, forming a vehicle for administering a medication, or conferring a therapeutic advantage on the active ingredient in the final dosage form, such as facilitating drug absorption, reducing viscosity, controlling the pharmacokinetics or enhancing solubility. Excipients can include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved as being acceptable for use in humans or domestic animals.

The composition can be manufactured using conventional formulation techniques. The present invention includes all of the usual forms. Solid and liquid compositions are preferred. Solid forms include crystalline forms, co-crystals (“coformers”), powders, tablets, capsules, lozenges (including liquid-filled), gels, liposomes, films, lipid nanoparticles and implantable polymers. Tablets include chewable tablets, buffering agents, and sustained release agents. Capsules include enteric coatings, lipid nanoparticles, emulsions, and sustained release compositions. Solid forms are for both oral use and reconstitution into solution. Solid forms include freeze-dried, lyophilized or photo-melted solids.

A compound of Formula (1a) or Formula (1b), or a pharmaceutically acceptable salt, crystal or co-crystal thereof, can be administered alone or in combination with or in conjunction with one or more other antiviral therapy. In embodiments, they will be administered as a formulation in association with one or more pharmaceutically acceptable excipients. Salts, carbohydrates, celluloses, buffering agents, sesame oil, cyclodextrans, Kleptose®, Captisol®, beta-lactones and other compounds may be used as excipients. The term “excipient” can refer to any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention (e.g., Formula (1a) and/or (1b)) and methods for their preparation will be readily apparent to those skilled in the art. Such compositions and methods for their preparation may be found, for example, in ‘Remington's Pharmaceutical Sciences’, 19th Edition (Mack Publishing Company, 1995). Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

In the solid composition, a compound of Formula (1a) or Formula (1b), or a pharmaceutically acceptable salt, crystal or co-crystal thereof, and, optionally, at least one additional antiviral therapy, can be in the dosage unit range. For example, a compound of Formula (1a) or Formula (1b), or a pharmaceutically acceptable salt, crystal or co-crystal thereof, can be administered in unit dosage ranges of 0.1-20 mg/kg/dose. Specific examples of dosages include 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, and 20 mg/mL. In embodiments, a compound of Formula (1a) or Formula (1b), or a pharmaceutically acceptable salt, crystal or co-crystal thereof, can be administered by established guidelines and, optionally, in combination with or in conjunction with another antiviral agent so as to target multiple virus pathways simultaneously.

Pharmaceutical compositions can include aqueous solutions, syrups, elixirs, emulsions, and suspensions. In the liquid composition, a compound of Formula (1a) or Formula (1b), or a pharmaceutically acceptable salt, crystal or co-crystal thereof, and another antiviral agent are present within a unit dosage range. For example, drug administration is in the unit dosage range of 0.1-20 mg/mL. Specific examples of dosages include 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, and 20 mg/mL. In general, other antiviral agents exist in a unit range similar to the clinically used class of drugs. For example, this can be 0.1-20 mg/mL.

A compound of Formula (1a) or Formula (1b), or a pharmaceutically acceptable salt, crystal or co-crystal thereof, and, optionally, at least one additional antiviral therapy, can be administered using any and all normal modes of administration. For example, embodiments can be administered using oral and parenteral (intramuscular, intravenous, subcutaneous injection, implantable) methods. In embodiments, the dosage regimen can be similar to other antiviral agents used clinically. Non-biodegradable polymeric implantable systems may include silicones, poly(urethanes), poly(acrylates) or copolymers such as poly(ethyelene vinyl acetate). Biodegradable polymeric implantable systems, may include poly(glycolic acid) (PGA), poly(caprolactone) (PCL), poly(lactic acid) (PLA), poly(lactic-co-glycolic acid) (PLGA), poly(amides), poly(anhydrides), poly(phosphazenes) or poly(dioxanone). The polymers may be blended by mixing polymers in selected proportions, or they may be tuned to release drug by selecting particular molecular weights. Some polymeric systems may include cellulose, chitosan and silk.

In embodiments, the dose for a compound of Formula (1a) or Formula (1b), or a pharmaceutically acceptable salt pharmaceutically acceptable salt, crystal or co-crystal thereof, can be 0.1-20 mg/kg body weight 1-7 times weekly. Many antiviral drugs require oral administration while newer antiviral drugs can be administered parenterally. However, the specific dosing regimen will be determined by the physician using the appropriate physician judgment.

The current invention describes a method wherein a compound of Formula (1a) or Formula (1b), or a pharmaceutically acceptable salt, crystal or co-crystal thereof, is administered in conjunction with at least one additional antiviral therapy. That is, a compound of Formula (1a) or Formula (1b), or a pharmaceutically acceptable salt, crystal or co-crystal thereof, can be administered in combination with or in conjunction with another antiviral drug useful in the treatment of a virus infection. Any drugs having a reducing action on viral loads can be used. In embodiments, nucleoside/nucleotide analogue reverse transcriptase inhibitors such as Abacavir (ABC), Didanosine (ddI), Emtricitabine (FTC), Lamivudine (3TC), Stavudine (d4T), Tenofovir (TDF), Zalcitabine (ddC) and Zidovudine (AZT); non-nucleoside/nucleotide reverse transcriptase inhibitors such as Delavirdine (DLV), Efavirenz (RFV) and Nevirapine (NVP); protease inhibitors such as Amprenavir (APV), Atazanavir (ATV), Indinavir (TDV), Ritonavir (RTV), Lopinavir/Ritonavir (LPV/RTV), Nelfmavir (NFV) and Saquinavir (SQV); fusion inhibitors such as Enfuvirtide (T20); or capsid inhibitors such as Lenacapvir (LCV) can be employed as conventional antiviral drugs which are already approved for use in HIV infection. In other embodiments, polymerase inhibitors, nonstructural protein 5A inhibitors, RNA-dependent RNA polymerase inhibitors, or DNA polymerase inhibitors can be employed as conventional antiviral drugs which are already approved for use in virus infections. In other embodiments, nirmatrelvir, ritonavir, remdesivir, molnupiravir, ribavirin, T-705, EIDD-1931, NITD008, Infergen®, enviroxime, pirodavir, acyclovir, adefovir, amantadine, ampligen, cidofovir, daclatasivir, ensitrelvir, foscamet, ganciclovir, letermovir, methisazone, moroxydine, nitazosanide, oseltamivir, peramivir, pleconaril, rimantadine, simeprevir, sofosbuvir, taribavirin, telaprevir, telbibudine, tromantadine, umifenovir, valaciclovir, valganciclovir, vidarabine, zanamivir can be employed as conventional antiviral drugs which are already approved for use in viral infections.

In preferred embodiments, a compound of Formula (1a) or Formula (1b), or a pharmaceutically acceptable salt, crystal or co-crystal thereof, can be administered in combination with or in conjunction with another antiviral therapy or in conjunction with cART. However, the method of the present invention is not limited thereto. In another embodiment, a compound of Formula (1a) or Formula (1b), or a pharmaceutically acceptable salt, crystal or co-crystal thereof, can be administered in combination with or in conjunction with or in conjunction with at least one additional antiviral therapy. In embodiments, the antiviral therapy can be an antiretroviral therapy. Non-limiting examples of such antiretroviral therapies comprise an HIV nucleoside/nucleotide reverse transcriptase inhibitor, an HIV non-nucleoside/nucleotide reverse transcriptase inhibitor, an HIV protease inhibitor, an HIV integrase strand transfer inhibitor, an HIV fusion inhibitor, an HIV entry inhibitor, an HIV virus budding or maturation inhibitor, an HIV capsid inhibitor or a broadly neutralizing antibody.

In combination with or in conjunction with another antiviral therapy, a compound of Formula (1a) or Formula (1b), or a pharmaceutically acceptable salt, crystal or co-crystal thereof, can be administered at dosages of between 0.1-20 mg/kg body weight one to seven times weekly over a period of 1 to 30 weeks. Specific examples of dosages include 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, and 20 mg/mL. Other drugs can be administered in amounts prescribed in antiviral therapy. The specific dosing regimen can be be determined using the appropriate physician judgment. The dose or pharmaceutically effective amount, administration route, the number of administrations, and the like of the drugs can be determined according to various conditions.

The present invention includes all pharmaceutically acceptable salt, crystal or co-crystal forms of a compound of Formula (1a) or Formula (1b), or a pharmaceutically acceptable salt thereof. Pharmaceutically acceptable salts are those in which the counter ion does not contribute significantly to the physiological activity or toxicity of the compound and functions as a pharmacological equivalent. In many cases, the salts have material properties that make them desirable formulations such as, for example, soluble or crystalline. The salts can be prepared according to conventional techniques using commercially available reagents. Examples include acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

The present invention also includes both unsolvated and solvated forms of a compound of Formula (1a) or Formula (1b), or a pharmaceutically acceptable salt, crystal or co-crystal thereof. Solvates do not contribute significantly to the physiological activity or toxicity of the compound but serve as a pharmacological equivalent. The term ‘solvate’ can refer to a molecular complex comprising the compound of the invention (e.g., a compound of Formula (1a) or Formula (1b), or a pharmaceutically acceptable salt, crystal or co-crystal thereof) and a stoichiometric amount of one or more pharmaceutically acceptable solvent molecules, for example, ethanol. The term ‘hydrate’ is employed when said solvent is water. Solvates can be formed in stoichiometric amounts, or from secondary solvents, or a combination of both. One type of solvate is a hydrate. Among the hydrated bodies include monohydrate, hemihydrate, and dihydrate.

While it is possible for the active ingredient (for example, the compound of Formula (1a) or Formula (1b), or a pharmaceutically acceptable salt, crystal or co-crystal thereof) to be administered alone, the active ingredient can be administered as a pharmaceutical composition. The composition, both for veterinary and for human use, can contain at least the compound of Formula (1a) or Formula (1b), or a pharmaceutically acceptable salt, crystal or co-crystal thereof, together with one or more acceptable carriers and optionally other therapeutic ingredients. In one embodiment, the pharmaceutical composition comprises a compound of Formula (1a) or Formula (1b), or a pharmaceutically acceptable salt, crystal or co-crystal thereof, a pharmaceutically acceptable excipient and a therapeutically effective amount of one or more (for example, one, two, three, or four; or one or two; or one to three; or one to four) additional therapeutic agents as defined herein. In some embodiments, the pharmaceutical composition comprises a compound of Formula (1a) or Formula (1b), or a pharmaceutically acceptable salt, crystal or co-crystal thereof, a pharmaceutically acceptable excipient and one other therapeutic ingredient. The carrier(s) are “acceptable” in the sense of being compatible with the other ingredients of the composition and physiologically innocuous to the recipient thereof.

In embodiments, the pharmaceutical compositions disclosed herein comprise a compound of Formula (1a) or Formula (1b), a pharmaceutically acceptable excipient and one other therapeutic agent selected from the group consisting of tenofovir, emtricitabine and dolutegravir. In some embodiments, the pharmaceutical compositions disclosed herein comprise a compound of Formula (1a) or Formula (1b), a pharmaceutically acceptable excipient and one other therapeutic agent selected from the group consisting of tenofovir, emtricitabine and dolutegravir. In some embodiments, the pharmaceutical compositions disclosed herein comprise a compound of Formula (1a) or Formula (1b), a pharmaceutically acceptable excipient and tenofovir. In some embodiments, the pharmaceutical compositions disclosed herein comprise a compound of Formula (1a) or Formula (1b), a pharmaceutically acceptable excipient and emtricitabine. In some embodiments, the pharmaceutical compositions disclosed herein comprise a compound of Formula (1a) or Formula (1b), a pharmaceutically acceptable excipient and dolutegravir.

The compositions can include those suitable for various administration routes. The compositions can conveniently be presented in unit dosage form and can be prepared by any of the methods well known in the art of pharmacy. Such methods include the step of bringing into association the active ingredient (for example, a compound of Formula (1a) or Formula (Ib) or a pharmaceutically acceptable salt, crystal or co-crystal thereof) with one or more inactive ingredients (for example, a carrier, pharmaceutical excipient, etc.). The compositions can be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21st Edition, Lippincott Williams and Wilkins, Philadelphia, Pa., 2006.

Still further, aspects of the invention are drawn to an antiviral composition comprising a first agent and a second agent, wherein the first agent comprises an antiviral therapy, and wherein the second agent comprises a compound according to Formula (1a) and/or Formula (1b)

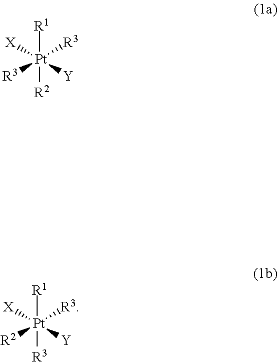

administering to a subject a compound according to Formula (1a) or Formula (1b),

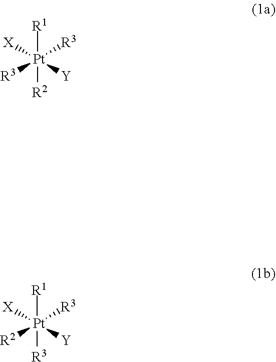

wherein X and Y are independently, nucleophilic exchangeable leaving groups, R¹ is NO₂ or OH.

R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OSO₂H OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl.

R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

In embodiments, the orientation of X and Y can be trans to one another. Platinum compounds of the cis configuration such as cisplatin, with adjacent chlorides, can crosslink DNA as alkylating agents, and are useful chemotherapeutics for the treatment of various cancers. Trans geometries of platinum compounds such as transplatin, have not been therapeutically effective for treating cancer, as the trans chloride geometries form fewer and different adducts with DNA than do cisplatin analogues. Pt(IV) prodrugs are largely intended to overcome systemic toxicities and become reduced to their corresponding Pt(II) cis-configuration geometries as cancer drugs. Pt(IV) oxidation states are preferred. Trans geometries of X and Y are generally preferred over cis geometries. Coordination ligands that are oriented approximately 180° (trans) around the central atom versus 90° (cis) result in greater perturbations to the approximately 109° (tetrahedral) coordination of the zinc atom and have demonstrated comparatively better activities in vitro.

Embodiments of the antiviral composition can further comprise a pharmaceutically acceptable salt, crystal, co-crystal, prodrug, or solvate thereof, or any combination thereof.

“Pharmaceutically acceptable salt” can refer to a physiologically or toxicologically tolerable salt, including but not limited to, when appropriate, pharmaceutically acceptable base addition salts and pharmaceutically acceptable acid addition salts. For example (i) where a compound contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see, for example, “Handbook of Pharmaceutical Salts: Properties, Selection and Use” by Stahl and Wermuth (Wiley-VCH, Weinheim, Germany, 2002).

The term “prodrug” can refer to a compound which is convertible in vivo by metabolic means (e.g. by hydrolysis, reduction or oxidation) to a compound of the invention. Suitable groups for forming prodrugs are described in ‘The Practice of Medicinal Chemistry, 2nd Ed. pp 561-585 (2003) and in F. J. Leinweber, Drug Metab. Res., 1987, 18, 379.

The compounds of Formula (1a) and/or (1b) can exist in both unsolvated and solvated forms. The term ‘solvate’ can refer to a molecular complex comprising the compound of the invention and a stoichiometric amount of one or more pharmaceutically acceptable solvent molecules, for example, ethanol. The term ‘hydrate’ can be employed when the solvent is water.

The compounds of Formula (1a) and/or (1b) can exist in any of the regulatory classifications of active pharmaceutical ingredient crystal or co-crystal solid-state forms. The crystal or co-crystal form can be considered in the same way as would any other morphic form such as a polymorph, solvate or hydrate. Co-crystals can be composed of two or more different molecules, typically an active pharmaceutical ingredient and an excipient, wherein the co-crystal can exhibit various physical properties affecting stability, bioavailability, or pharmacokinetics.

Where compounds used in the antiviral compositions exist in one or more geometrical, optical, enantiomeric, diastereomeric and tautomeric forms, including but not limited to cis- and trans-forms, E- and Z-forms, R-, S- and meso-forms, keto-, and enol-forms, then, unless otherwise stated, a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

The antiviral compositions described herein can be administered by any suitable therapeutic method and technique presently or prospectively known to those skilled in the art. For example, antiviral compositions as described herein can be administered by any suitable route known in the art including, for example, oral, nasal (e.g., via aerosol inhalant), rectal, ex vivo (reintroduction of treated tissues), and parenteral routes of administration. As used herein, the term parenteral includes topical, subdermal (e.g., as in an implant), subcutaneous, intradermal, intravenous, intramuscular, intraperitoneal, and intrasternal administration, such as by injection. Administration of the subject platinum compounds of the invention can be continuous or at distinct intervals as can be readily determined by a person skilled in the art.

In embodiments, the antiviral composition can be administered utilizing liposome technology, antibody-conjugation, peptide-conjugation, nanotechnology (such as carbon nanotubes, gold nanospheres, or nanoslow-release capsules), polymeric sugars, electroporation, implantable pumps, and biodegradable containers. Certain of these delivery methods can, advantageously, provide a uniform dosage over an extended period of time while others provide immediate and/or local targeting. The platinum compounds of the present invention can also be administered in their salt derivative forms or crystalline forms known to those of ordinary skill in the art.

In embodiments, the antiviral composition can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. For example, the antiviral compositions as described herein can be formulated such that a bioeffective amount of the platinum compound is combined with a suitable carrier in order to facilitate effective administration of the composition. The antiviral compositions used in the present methods can also be in a variety of forms. These include, for example, solid, semi-solid, and liquid dosage forms, such as tablets, pills, powders, liquid solutions or suspension, suppositories, injectable and infusible solutions, aerosol particle, and sprays. The preferred form depends on the intended mode of administration and therapeutic application. The compositions can also include conventional pharmaceutically acceptable carriers and diluents which are known to those skilled in the art. Examples of carriers or diluents for use with the subject platinum compounds include ethanol, ethyl acetate, dimethyl sulfoxide, glycerol, alumina, starch, and equivalent carriers and diluents. To provide for the administration of such dosages for the desired therapeutic treatment, pharmaceutical compositions of the invention will advantageously comprise between about 0.1% and 99%, and especially, 1 and 15% by weight of the total of one or more of the subject platinum compounds based on the weight of the total composition including carrier or diluent.

The antiviral compositions described herein and methods for their preparation will be readily apparent to those skilled in the art. Such compositions and methods for their preparation may be found, for example, in ‘Remington's Pharmaceutical Sciences’, 19th Edition (Mack Publishing Company, 1995). Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition. Lippincott, Williams & Wilkins, Philadelphia, Pa., (2005), at p. 732, Table 38-5, both of which are hereby incorporated by reference herein.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims, and those skilled in the art will understand how to make changes and modifications to the disclosed embodiments to meet their specific requirements or conditions. Changes and modifications may be made without departing from the scope and spirit of the invention. It is understood that use of the singular embraces the plural and vice versa. In addition, the steps of any method described herein may be performed in any suitable order and steps may be performed simultaneously if needed.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Lentivirus replication in compartmental tissue reservoirs, for example lymphatic tissues, bone marrow and the central nervous system, continue despite effective cART suppression in blood plasma. Such viral reservoirs are commonly denoted as sanctuary sites, where distinct viral pathologies may evolve. Those of the central nervous system comprise infections to macrophages, astrocytes and microglial cells, resulting in inflammation which damages neurons [J Hokello 2021]. Continuing active virus replication has been reported in lymphatic tissues (lymph nodes, spleen, e.g.) despite multiple years of undetectable viremia measured in blood plasma [A M Cadena 2021; E. Scholz 2021; R Lorenzo-Redondo 2016; Dionna W Williams 2016]. Comparative frequencies of intact, defective, and inducible proviruses between peripheral blood and lymph node CD4⁺ T cells indicate that this discrepancy is not due to differences between cellular origins of virus in these compartments [A R Martin 2021]. Nonetheless, secondary lymphoid organs (lymph nodes, spleen, e.g.) have been identified as primary sources of both rebounding virus and of persistent virus replication, even after years of successful plasma viremia suppression with cART, in both monkeys [J D Estes 2017; Siddiqui 2019] and humans [W R McManus 2019]. Thus, reducing sources of virus in viral reservoirs, including the diverse cell subpopulations with integrated proviral DNA, and virus replication in compartmental tissue reservoirs (lymph nodes, spleen, e.g.) remain obstacles for current therapeutic management.

Virtually all FDA-approved HIV therapeutics are designed to disrupt the virus replication cycle to achieve clinical suppression of plasma viremia (<40 copies/mL clinical limit of detection). If, however, therapy is interrupted, virus rebound typically occurs within two weeks. Therapeutic management is a lifelong challenge and commitment. Thus, new therapeutic strategies for treating HIV infection have focused on reducing sources of persistent virus. While “sterilizing” approaches have been successful in only five known cases with CCR5Δ32/Δ32 bone marrow or stem cell transplants to date, this outcome is considered impractical for most PLWH. “Functional” or long-term remission approaches are being sought, with a view to reducing the viral burden residing in viral reservoirs, and by enhancing antiviral immune responses. These approaches differ from “long-acting” formulations of drugs, which reduce the dosing frequency while maintaining effective circulating drug concentrations through slow-release Such therapeutic strategies would provide relief from continuous drug burdens and reduce comorbidities and mortalities associated with HIV infection.

Current standard of care (cART) has limitations:
It is not curative.
Increased risks of resistant variants and the transmission of ART drug resistance to broader populations as more individuals initiate cART;
Continuous therapeutic adherence requirements, i.e. a “Sword of Damocles”;
Management of side effects, especially given that comorbidities are involved;
Age-related morbidities lead to a general decline in health and require additional drugs and contraindications;
Failure to reduce the residual provirus in blood and tissue reservoirs;
Lack of suppression in physiological reservoirs, leading to compartmental persistence and potential evolution of virus;
Threat of reseeding the periphery and relapse upon cART interruption or failure;
Increased morbidities despite virus suppression, e.g. HIV-Associated Neurocognitive Disorders (HAND), nonalcoholic fatty liver disease, premature aging, cardiovascular and kidney diseases;
The genetic diversity of HIV-1, high mutational rate and integration with host DNA are therapeutically challenging;
Vaccines have thus far demonstrated limited protection against HIV-1 infection;
High costs associated with treatment management;
Complexities of polypharmacy; and
Access to appropriate care and availability of drugs for adequate treatment continues to exacerbate the developing world.

There are several classes of FDA-approved antiretroviral drugs (ARVs), most of which target early steps in the virus replication cycle. Nucleoside RT inhibitors (NRTIs) and non-nucleoside RT inhibitors (NNRTIs) target viral reverse transcriptase either by binding directly to an allosteric site on the enzyme (NNRTIs) or providing a chain-terminating substrate during reverse transcription (NRTIs). Integrase strand transfer inhibitors (INSTIs) bind the integrase-viral DNA complex, effectively blocking viral DNA integration into the host genome. Protease inhibitors (PIs) act late during the virus replication cycle by blocking the proteolytic cleavage of the viral polyprotein precursors, obstructing virus particle maturation. Other antiviral agents targeting cell binding and entry, maturation, and viral structural proteins (capsid protein, e.g.) continue to be developed. Current therapeutic standards of care for PLWH require multiple drugs of different classes (cART) or ARVs in combination with broadly neutralizing antibodies to suppress virus replication, reducing the viral load (plasma viremia, measured as RNA copies/mL) to levels that are extremely low in clinical assays (<40 RNA copies/mL). However, current HIV therapy is not curative and is required for a lifetime.

In addition to HIV-related lentiviruses, other viruses utilize zinc finger domains for critical viral protein activities. Non-limiting examples of such viruses include other Retroviruses [A. Monette 2020], Coronaviruses, Paramyxoviruses, Togaviruses, Flaviviruses, Bunyaviruses, and Hepadnaviruses. Some of these virus families/orders pose serious risk potentials for future pandemics. The therapeutic benefits of this invention targeting zinc finger domains demonstrates unique long term antiviral activities in the absence of continuing therapy and a good safety profile to support utilities as a broad-spectrum antiviral small molecule for diverse current and emerging virus infections.

### Example 2

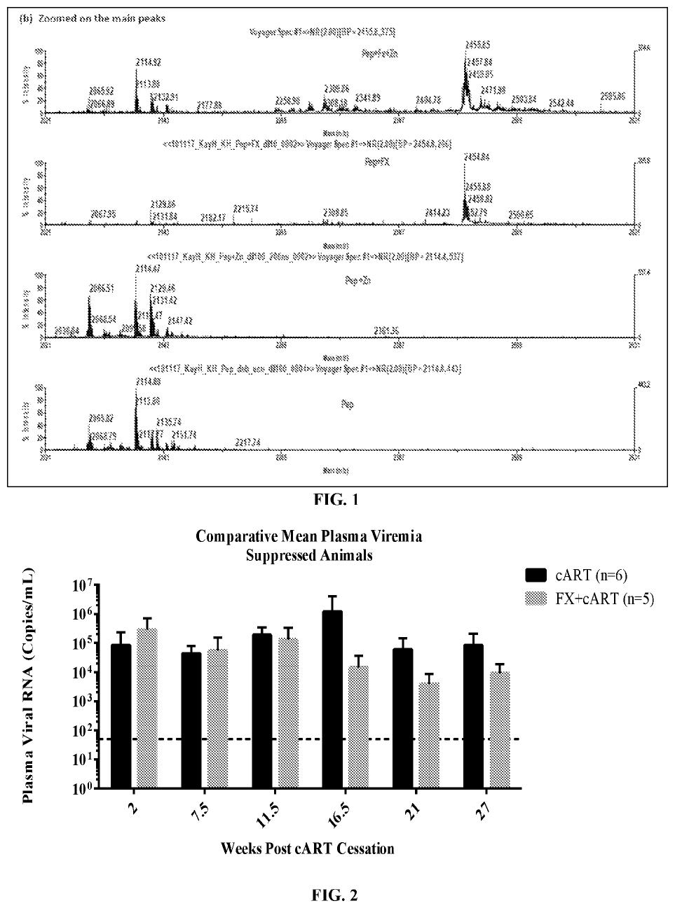

The nucleocapsid protein (NC) of HIV is an established but elusive therapeutic target for HIV infection. Currently available antiretroviral drugs predominately target viral enzymes involved in replication, including reverse transcriptase, integrase and protease. Recently, a new drug class targeting the structural capsid protein has been approved. NCp7 is a small (55 residues) structural protein of HIV containing two zinc-binding domains, resulting from a series of protease cleavages of the translated Gag polyprotein [A Mouhand 2020]. One of the unique attractions of the NC target is its direct association with viral nucleic acids as a chaperone, although the nucleocapsid protein has multiple functions through the life cycle of the virus. These activities are directly related to the two critical zinc-binding domains of NC [Y Wang 2021]. Each of four amino acid residues coordinates to a zinc atom through highly conserved cysteine and histidine residues. Disruption of the zinc coordination to these residues alters the required tetrahedral functional geometry of the associated protein(s), which can be achieved using zinc chelators or zinc ejectors, such as S-acyl-2-mercaptobenzamide thioesters (SAMT), ethylenediaminetetraacetic acid (EDTA), disulfiram, Epidithiodiketopiperazines (ETP), dithiazoles, thiaselenasoles, nitrosobenzamides, dithiobisbenzamide disulfides, benzisothiazolones, pyridinioalkanoyl thioesters, 2-methyl-3-phenyl-2H-[1,2,4]thiazol-5-yideneamine (WDO-217), N,N′-bis(4-ethoxycarbonyl-1,2,3-thiadiazol-5-yl)benzene-1,2-diamine (NV038). Unlike zinc chelators or zinc ejectors, the compounds of this invention have been shown to bind to the nucleocapsid protein zinc domain in vitro, both in the absence and in the presence of zinc (FIG. 1), and thus can compete with zinc, resulting in a different orientation of the protein tertiary structure. Zinc is also coordinated by other HIV proteins, including HIV-1 integrase, viral infectivity factor (Vif), Trans-Activator of Transcription (Tat), and HIV-2 virion-associated protein (Vpx). The in vivo safety and efficacy testing of targeting this viral motif by compounds of this invention support long term antiviral activities, even in the absence of continuing treatment.

### Example 3

Application to Other Virus Infections, including viral protein, host protein, and viral protein-host protein and viral protein-nucleic acid interactions.

Viral proteins comprising zinc binding domains are expressed by multiple virus families in diverse functions and are thus recognized as therapeutic targets [C C Garcia and E B Damonte 2007; C Abbehausen 2019]. This target profile consists of a specific sequence of amino acids—usually cysteine and histidine—that coordinate a zinc atom, being strictly conserved and less prone to mutational resistance than other viral targets. Virus mutations lead to continuing drug, antibody and vaccine resistance. Viruses mutate, creating continual therapeutic challenges.

Viruses known to utilize zinc binding domains include those of the Coronaviridae family. Viruses of this family include but are not limited to Severe Respiratory Syndrome Corona Virus 2 (SARS-CoV-2), Middle East Respiratory Syndrome (MERS-CoV) and SARS-CoV-1. Both viral and host proteins containing zinc binding domains have been identified as desirable therapeutic targets determining the severity of the disease [S Esposito 2022]. Identified host protein targets regulate transcription factor activities and antiviral activities in viral infection, including but not limited to: zinc finger antiviral protein (ZAP, a mammalian host restriction factor); zinc finger NFX1-type containing 1 (ZNFX1); zinc finger CCHC-type containing 3 (ZCCHC3); Krüppel-like factor 2 (KLF2); MADP1 Zinc finger CCHC-type and RNA-binding motif 1 (hnRNP1); zinc finger DHHC domain-containing (ZDHHC); and the zinc finger DHHC domain-containing (ZDHHC) palmitoyl transferase proteins, including the multiple ZDHHCs palmitoylating different sites and located in different cellular compartments. Cysteine (C) and histidine (H) residues can coordinate to zinc in these zinc domains (ZD), where the sites are sometimes characterized by the coordinating residues, such as CCHC comprising three cysteines and one histidine in the order given.

Coronavirus zinc finger protein targets include: papain-like protease (PLpro); 3C chymotrypsin-cysteine-like peptidase or main protease (Mpro) [CP Delgado 2022]; non-structural replicase protein 14 (nsp14); non-structural replicase protein 10 (nsp10); non-structural replicase protein 13 (nsp13); non-structural replicase protein 2 (nsp2); and non-structural replicase protein 12 (nsp12). High conservation of these targets is shared across the Coronaviridae family.

Importantly, most viruses have developed tools to evade cellular immune responses. Accordingly, virus protein—host protein interactions may interfere with the antiviral response mechanisms of the host cells [G Wang and C Zheng 2020]. For instance, PLpro is foremost a viral protease, but it also deubiquitinates and deISGylates, deconjugating ubiquitin and interferon-stimulated gene product 15 (ISG15) from their substrate signaling proteins, thus attenuating host type I interferon responses [S Esposito 2022]. Indeed, SARS-CoV-2 and HIV-1 share some molecular pathways involved in inflammation, immune response, and cell cycle regulation, indicating a number of virus-host interactions common to both virus pathologies [O Tarasova 2020]. The lentivirus nucleocapsid protein is known to interact with multiple host cell proteins [J Klingler 2020].

In addition to other Retroviruses and members of the Coronaviridae family, other members of virus families utilizing target zinc finger domains include, but are not limited to: Paramyxoviridae, Togaviridae, Flaviviridae, Bunyaviridae, and Hepadnaviridae.

Fourteen genera of viruses comprise the Paramyxoviridae family, which express viral V accessory proteins containing zinc binding domains. Members within this family are negative, single-stranded RNA viruses infecting humans, mammals and fish. Example members of the Paramyxoviridae family include measles virus (MeV), Nipah virus (NiV), mumps virus (MuV), Sendai virus (SeV); Parainfluenza virus 5 (PIV-5); Human parainfluenza virus 2 (HPIV-2); Hendra virus (HeV); and Newcastle disease virus (NDV). Viral V proteins are also structural components of the nucleocapsid core for some, but not all paramyxoviruses. Nonetheless, conserved cysteine residues with one histidine residue have been shown to coordinate zinc for V protein activities. In order to subvert host cell immune responses, viral V proteins interact with host cell melanoma differentiation associated protein 5 (MDA5); interferon regulatory factor 3 (IRF-3); retinoic acid-inducible gene-I (RIG-I); TRIM25 ubiquitination and signal transduction and activators of transcription (STAT) proteins to block host interferon signaling through mechanisms including degradation, inhibition of phosphorylation and inhibition of translocation [M D Audsley 2013; M T Sanchez-Aparicio 2018; S Uchida 2018; T R Keiffer 2020]. Thus, the V proteins are recognized therapeutic targets in this virus family.

Chikungunya virus (CHIKV) and Sindbis virus (SINV) are members of the Alphavirus genus belonging to Togaviridae family and emerging threats to world health, transmitted by mosquitos. The nonstructural protein 3 (nsP3) includes the alphavirus unique domain (AUD), which is a recognized therapeutic target. The AUD coordinates zinc. Mutations to the residues adjacent to the zinc-binding cysteines reveal an essential role for nsP3 in virus replication [Y Gao 2019].

Dengue Viruses 1-4, West Nile Virus, Japanese encephalitis virus, Zika and Yellow Fever Virus are members of the Flaviviridae family. Structure-based sequence alignment indicates key cysteine and histidine residues coordinating zinc ions that are shared across these viruses. These residues are conserved in the polymerase of all four Dengue serotypes and in the RNA-Dependent RNA polymerase (RdRp) of Yellow Fever Virus. Minor differences exist for West Nile and Japanese encephalitis virus, but these also utilize key viral proteins coordinating zinc [T L Yap 2007]. The zinc chelator, N,N,N′,N′-tetrakis(2-pyridinylmethyl)-1,2-ethanediamine (TPEN), inhibits dengue virus and Japanese encephalitis virus (JEV) infection. Zinc chelation affects both early and late stages of the infectious cycle in addition to eliciting an antiviral response through the induction of interferon signaling [M Kar 2019]. Thus, disrupting the zinc finger domain is a potent antiviral strategy against flaviviruses.

Crimean Congo Hemorrhagic Fever virus (CCHFV) is an arbovirus, member of the Orthonairovirus genus, order Bunyavirales, causing hemorrhagic fever. There are currently limited viruses expressing zinc fingers on surface viral envelope glycoproteins, but CCHFV is one of these [DF Estrada 2011]. Four of the five genera of Bunyavirales share a conserved dual CxCxHxC motif of cysteine (C) and histidine (H) residues with x representing other residues. Zinc is required for the proper folding of the CCHF virus protein, as determined by chelating the zinc using ethylenediaminetetraacetic acid. Other genera of the Bunyaviridae family known to utilize viral zinc finger domains include Hantaviruses, Peribunyaviridae and Arenaviruses. Viruses in this order that cause disease in humans include, but are not limited to, California encephalitis virus, Rift Valley Fever virus, Lassa fever virus, Argentine hemorrhagic fever (also known as Junin) virus and lymphocytic choriomeningitis mammarenavirus.

Hepatitis B Virus and other mammalian hepadnaviruses express an X protein (HBx) to overcome host cell restriction factors suppressing HBV transcription. A conserved CCCH motif binding zinc in the HBx protein is required for this viral activity [D Ramakrishnan 2019].

### Example 4

Molecules of this invention demonstrate long term reduction of virus burdens in the absence of continuing therapeutic treatment. Additionally, virus-producing cells in anatomical reservoirs harboring persistent virus are reduced, mitigating the burdens associated with a chronic infection. Viral reservoirs in lymphatic tissues continue to produce virus despite full suppression of virus replication in blood under the current standard of care therapies for HIV infection. The profound antiviral impacts of targeting zinc finger domains establish it as an “Achilles Heel” for therapeutic intervention. The invention described herein represents a broad antiviral strategy addressing current and future therapeutic needs for a broad range of virus infections.

Animal models useful for studying lentivirus and other virus infections have been developed for obvious necessity. Example models for HIV infection include the FIV/cat, SIV/SHIV/nonhuman primate and humanized mouse models [T Hatziioannou and D T Evans 2012; B B Policicchio 2016; A J Kleinman 2022]. While each model has value, the SIVmac-infected Rhesus macaque model is used for studying “curative” and long-term remission therapeutic strategies for HIV infection. This is because of similarities between SIV and HIV provirus integration in target cells, shared viral latency maintenance and latency activation signaling pathways, and similar latent reservoirs [A J Kleinman 2022; G Terrade 2021]. Additionally, nonhuman primate (NHP) models provide safety, pharmacokinetic and immunologic measures as a preclinical model for translation to human clinical trials [K K A VanRompay 2017]. A sex difference between male and female Rhesus macaques has been reported in multiple SIV/SHIV studies, with males being the more challenging to elicit adaptive responses as compared with females for the purposes of humoral and vaccine control [L K Miller-Novak 2018].

The SIVmac251 strain of lentivirus was derived from an Indian-origin rhesus macaque that developed a B-cell lymphoma from SIV stocks that had been passaged four to five times in human PBMC. The Rhesus macaque model is an established and commonly used vaccine and virus challenge model that has also been used to evaluate HIV “curative” therapeutic strategies [R M Dunham 2013; J B Whitney 2014]. While Indian and Chinese-origin Rhesus macaques (Macaca mulatta) have different pathological responses to the same virus strains, SIVmac251 replicates to high levels and quickly causes disease in macaques. A median survival of ˜72 weeks has been reported in this model.

Generally, Rhesus macaques will reach peak viral loads by week 2 post infection and establish viremia set point by week 12; intrarectal inoculation ensures a pre-viremic eclipse phase of viral replication in mucosal and lymphoid tissues, consistent with human routes of infection. The viral reservoir is seeded within 3 days following intrarectal SIVmac251 infection [J B Whitney 2014]. Peak viral loads during acute infection are typically 10⁷-10⁹ RNA copies/mL, reducing to 10⁴-10⁷ over subsequent weeks. The rate of disease progression for SIV-infected macaques is considerably more rapid than for HIV-1-infected humans; Indian-origin Rhesus macaques typically progress to AIDS within 1-2 years of SIV infection, compared with 8-10 years for humans who are infected with HIV and not receiving antiretroviral therapy. Furthermore, 20-30% of animals rapidly progress toward AIDS-like disease. Aspects of the invention translate discoveries made from an SIV/NHP preclinical model study. These results further support translational treatment of lentivirus and other virus infections in humans; a good preclinical safety profile as a monotherapy and in combination with other antiviral drugs; long term reductions in viral setpoints; reductions of virus in lymphatic tissues; and a host target or virus-host protein interaction target benefit that may be shared across a broad range of virus infections. The present invention could be useful to support long term remission from continuous antiretoviral drugs for PLWH.

Twenty-four specific pathogen-free (SPF), all male, Rhesus macaque (Macaca Mulatta) nonhuman primates (NHPs) of Indian origin were utilized for this study. The animals were tested to be free from simian type D retrovirus (SRV), simian immunodeficiency virus (SIV), simian T-cell lymphotropic/leukemia virus (STLV) and Cercopithecine herpesvirus 1 (CHV-1) at study initiation. All animals were pre-screened as negative for the protective MHC class I alleles Mamu-A*01, Mamu-B*08, and Mamu-B*17 since these alleles are associated with elite control of SIV infection. All animal studies were approved by the appropriate Institutional Animal Care and Use Committee (IACUC).

Inoculation of NHPs with SIVmac251. Animals were intrarectally challenged with 500 Tissue Culture Infectious Dose (TCID)₅₀ of SIVmac251 challenge stock. Virus inoculum was introduced into the rectum by bolus injection under ketamine anesthesia. Each animal was subsequently monitored.

The twenty-four (24) SIVmac251-infected Rhesus macaques were divided into four groups: (1) sham-treated controls (vehicle only); (2) FX101-treated; (3) combination antiretroviral therapy (cART)-treated and (4) FX101 plus cART-treated. Animals were semi-randomly assigned to each group to counterbalance individual differences in animal size, age, viral loads and CD4⁺ T-cell counts as practicable. Comparative evaluations between groups were determined to evaluate whether FX101 alone and/or in combination with cART safely reduces virus in plasma, CSF, tissue reservoirs and/or sanctuary sites. The primary objective of this study was to compare virus burden measures between treatment groups of SIV-infected animals, including the persistence of viral reservoirs. Comparative longitudinal clinical measures were assessed to evaluate the safety profile.

Antiviral treatments were initiated at 10 weeks post-infection after the initial inflammatory response had subsided. All drug treatments were administered subcutaneously to avoid repeated sedations of the animals. The control animals received vehicle only (10% dimethylsulfoxide in phosphate buffered saline, DMSO/PBS). The dual-treated group received equal dosing amounts and frequencies of FX101 (3.0 mg/kg, 3× weekly) and cART (daily); the cART treatment group received two nucleo(t/s)ide RT inhibitors (NRTIs), emtricitabine (FTC, 50 mg/kg) and tenofovir disoproxil fumarate (TDF, tenofovir prodrug, 5.1 mg/kg) plus one integrase strand transfer inhibitor (INSTI), dolutegravir (DTG, 2.5 mg/kg). This cART regimen has demonstrated to be effective in the SIV model [G Del Prete 2015] and is translationally relevant to currently prescribed HIV therapeutic regimens. Therapeutic cART treatments were administered for up to 24 total weeks, with animals followed for up to 27 weeks post cART cessation (˜1 year post infection). All animals were monitored daily for signs of adverse events during the study.

Collection of blood, CSF and urine. Femoral blood, cisternal cerebrospinal fluid (CSF) and urine samples were collected from macaques under sedation (ketamine, 10 mg/kg) at specific time points. Approximately 1.0 mL of CSF was collected from the cisterna magna by gravity flow at selected times. CSF samples collected on ice were clarified by centrifugation to pellet cells. The cell-free CSF was frozen at −80° C. in multiple aliquots to avoid repeated freeze-thaw cycles. Urine samples were collected for urinalysis and for drug clearance pharmacokinetics. Blood was collected for complete blood count (CBC) with white blood cell differential, blood serum chemistry and urinalysis (UA) (performed by IDEXX laboratories). Blood plasma was collected from EDTA-anticoagulated blood collections (Sarstedt Monovettes™) following centrifugation. Peripheral blood mononuclear cells were isolated using Ficoll separation with SepMate™ tubes (StemCell™ Technologies) using the manufacturer's protocols, then washed twice with phosphate buffered saline before freezing at −80° C. in aliquots.

Four of the 24 animals were eliminated from the study, reducing the number of animals in each study arm. Eliminated animals were either rapid progressors, spontaneous controllers and/or animals that developed secondary infections. Since the cohorts were staggered over time, with not all animals becoming infected on the first virus inoculum, animals were redistributed as possible to balance groups prior to initiation of treatment.

### Example 5

Cotherapeutic Treatment of FX101+cART (“FX”+cART) Results in Lower Viremia Set Points as Compared with Treatment with cART, Post Treatment Cessation (FIG. 2-6).

Determination of plasma and CSF Viral RNA Levels. The amount of RNA copies per ml was determined using a TAQMAN assay. The assay utilized primers and a probe specifically designed to amplify and bind to a conserved region of the Gag gene of SIV. The assay has been designed to quantitate a large spectrum of SIV and SHIV isolates. The amplified signal was compared to a known standard curve to provide RNA copies per ml for plasma and CSF.

Procedure: A volume of 0.2 ml of Sample was added to 0.2 ml of AL lysis buffer with carrier RNA. A protease volume of 20 μl was added and then incubated at 56° C. degrees for 15 minutes. Next, the sample was centrifuged at 11,000×g for 1 minute and washed with wash buffer. The sample was centrifuged, washed with wash buffer, centrifuged again, and washed with absolute ethanol. The sample was finally centrifuged and re-suspended in 50 μl of buffer.

The primer-probe set covers a highly conserved region in GAG and is present in all strains of SIV. The sequences for the SIV primer/probe sets are as follows:

Primers:
SIV-Forward:
GTCTGCGTCATCTGGTGCATTC
SIV-Reverse:
CACTAGGTGTCTCTGCACTATCTGTTTTG
Probe: SGAG23:
6FAM-CTTCCTCAGTGTGTTTCACTTTCTCTTCTGCG-TAMRA

RNA controls: To make the RNA control, stock SIV was processed following the same procedure described above. To determine the amount of RNA, an O.D. reading at 260 was taken: 1.0 OD at A260=40 μg/ml of RNA. With the number of bases known and the average base of RNA weighing 340.5 g/mole, the number of copies was then known and the control diluted accordingly. The final dilution of 10⁷ copies per 3 μl was divided into single use aliquots of 10 μl. These were stored at −80° C. until needed. Several aliquots were chosen at random and compared to previous controls to verify consistency.

Master Mix preparation: 2.5 ml of 2× buffer containing Taq-polymerase obtained from the TAQMAN RT-PCR kit (Bioline USA Inc., Boston, MA) was added to a 15 ml tube. Then, 50 μl of the RT and 100 μl RNAse inhibitor from the kit was added. The primer pair at 2 μM concentration was also added in a volume of 1.5 ml. Lastly, 0.5 ml of water and 350 μl of the probe at a concentration of 2 μM were added. The tube was vortexed.

Reactions: 47 μl of the master mix and 5 μl of the sample RNA were added to each well in a 96 well plate. All samples were tested in triplicate, and the plates were sealed with a plastic sheet.

Control curve preparation: Samples of the control RNA were obtained from the −80° C. freezer. The control RNA had been prepared to contain 10⁷ copies per 3 μl. Seven 10-fold serial dilutions of control RNA were prepared using RNAse-free water by adding 5 μl of the control to 45 μl of water and repeating this for 6 dilutions. This resulted in a standard curve with a range of 1 to 10⁷ copies/reaction. Triplicate samples of each dilution were prepared. The sample was diluted as needed if the copy number exceeded the upper limit of detection.

Amplification: The plate was placed in an Applied Biosystems 7500 Sequence detector and analyzed using the following program: 48° C. for 30 minutes, 95° C. for 10 minutes followed by 40 cycles of 95° C. for 15 seconds and 1 minute at 60° C. The number of copies of RNA per ml was calculated by extrapolation from the standard curve and multiplied by the reciprocal of 0.5 ml extraction volume, i.e., 20. This supports a practical range of 50 to 5×10⁷ RNA copies per ml. The intra-assay variation of this test is around 0.3 Log.

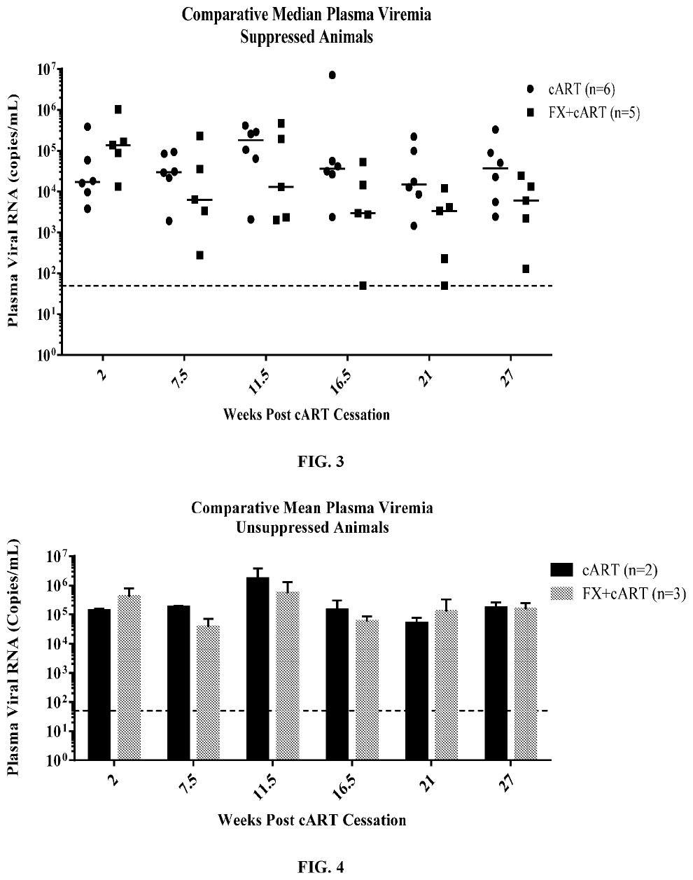

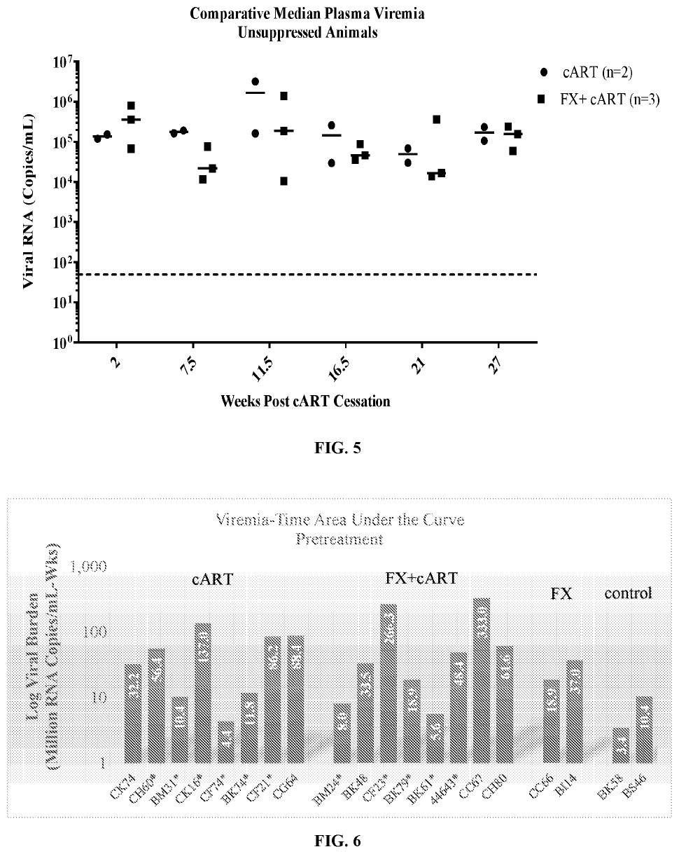

Monotherapeutic FX101 (“FX”) at doses of 3.0 mg/kg 3 times weekly had no apparent direct virus replication inhibiting activities in this SIV/NHP model during the treatment period, as measured by real time polymerase chain reaction (RT-PCR) plasma viremia over multiple sample collection time points during the treatment period. Importantly, some FDA-approved HIV antivirals are not always effective for lentiviruses in nonhuman primate models, such as SIVcpz (chimpanzee) viruses that use infection pathways escaping the antiviral activity of the capsid inhibitor PF74, which “depends on cellular cofactors” [AP Twizerimana 2020]. Most non-nucleoside/tide reverse transcriptase inhibitors are specific to the virus. Despite lack of apparent virus replication inhibition in peripheral blood, long term antiviral effects of cotherapeutic FX+cART became evident commencing approximately 16 weeks post cART treatment cessation for animals having achieved a relatively short period (2-8 weeks) of virus suppression (<50 copies/mL, limit of detection) with cART cotherapy (FX+cART group). Comparative viremia level set points (steady state dynamics) for the FX+cART cotherapeutic group continued through Week 27 post cART treatment cessation (˜1-year post infection at study termination), demonstrating nearly a log difference between group mean (and median) viremias over these 10.5 weeks measured over three consecutively scheduled sample collections (Tables I and II; FIG. 2 and FIG. 3). It is noteworthy that one of the six FX+cART cotherapeutic animals controlled to undetectable viremia (<50 copies/mL) at two collection time points, in the absence of any continuing therapeutic treatment. Animals not achieving a period of therapeutic virus suppression exhibited higher viremia set points and no differences between comparative treatment groups (FIG. 4 and FIG. 5). Pretreatment virus burdens, as measured by area under the curve over this time period (weeks 0-10 post infection, pretreatment), illustrate that the cotherapeutic FX+cART group demonstrated equal or greater pretreatment viral burdens as compared with cART group animals (FIG. 6). Together, these results indicate that adjunctive administration of FX101 together with cART standard of care leads to a reduction in the persistent viral burden, evidenced by lower setpoints off therapy in the cotherapeutically treated animals. Since this outcome was associated with animals achieving a period of virus suppression, longer periods of virus suppression may provide long term remission from the requirement of continuous cART.

Notably, the majority of published SIV/NHP long term remission studies required a full year of cART to achieve long term plasma viremia suppression or to affect rebound virus kinetics or to reduce viral reservoirs. The SIVmac251/NHP model can require 20 weeks of cART (and even up to 40 weeks when cART commences after a year of infection) to achieve virus suppression<50 RNA copies/mL [E N Borducchi 2016; DH Barouch 2013; A A Okoye 2018; P T Liu 2020]. The current study was originally designed for only 15 total weeks of cART treatment so as to allow a post treatment monitoring period. Animals exhibiting >5×10⁶ RNA copies/mL at 9 weeks post infection proved to be the most difficult to suppress at any point during the drug treatment period, and such animals have been identified as “unsuppressed”, having no time point sample collection measuring <50 RNA copies/mL during the drug treatment period. “Suppressed” animals are defined as having at least one sample time point during therapeutic treatment measuring <50 RNA copies/mL (the PCR limit of detection). Total periods of full virus suppression ranged between 2-8 weeks for animals in this study, a fraction of that achieved in other studies in this model (c.f.≥1 full year), emphasizing these remarkable outcomes. These outcomes support a corresponding reduction in the size of the viral reservoir, lower levels of inflammation, reductions in associated comorbidities and improved long term prognoses. Without wishing to be bound by theory, one cotherapeutic benefit of FX101 is the reduction of the viral reservoir when a subject is virologically suppressed, thus reducing the viremia setpoint in the absence of continuing therapy.

Longer periods of complete virus suppression are anticipated to result in greater beneficial outcomes. Animals achieving periods of virus suppression (<50 RNA copies/mL) also exhibited higher frequencies of CD4⁺ T cells, as measured by flow cytometry, thus protecting against the loss of this important immune cell and its functions (FIG. 19-22). Protecting against the loss of CD4⁺ T cells with cART may be particularly beneficial in combination with FX101. No differences between viral load setpoints were observed in comparative groups of unsuppressed animals, further exhibiting higher plasma viremias. Continuing cART after discontinuing FX101 may also provide further therapeutic benefit since discontinuing cART initially resulted in higher viremias at 2 weeks post treatment cessation for the FX+cART group.

TABLE I
Mean Plasma Viremia (SIV RNA copies/mL)
“Suppressed” Animals

| Weeks Post | | | | | |
|---|---|---|---|---|---|
| Treatment | | | | FX + cART Group | |
| Cessation | P | cART Group (n = 6) | | (n = 5) | |
| Time (wks) | value | Mean | SEM | Mean | SEM |
| 2 | 0.29 | 81,807 | 60,957 | 284,980 | 185,569 |
| 7.5 | 0.79 | 43,570 | 14,963 | 55,350 | 44,371 |
| 11.5 | 0.64 | 188,197 | 63,821 | 135,874 | 90,549 |
| 16.5 | 0.38 | 1,209,548 | 1,178,112 | 14,590 | 9,848 |
| 21 | 0.19 | 59,748 | 35,215 | 3,935 | 2,152 |
| 27 | 0.22 | 83,145 | 50,903 | 9,178 | 4,388 |

TABLE II
Mean Plasma Viremia (SIV RNA copies/mL)
Unsuppressed Animals

| Weeks Post | | | | | |
|---|---|---|---|---|---|
| Treatment | | | | FX + cART Group | |
| Cessation | P | cART Group (n = 2) | | (n = 3) | |
| Time (wks) | value | Mean | SEM | Mean | SEM |
| 2 | 0.394 | 136,000 | 17,000 | 410,833 | 214,064 |
| 7.5 | 0.015* | 178,500 | 14,500 | 36,467 | 19,886 |
| 11.5 | 0.432 | 1,686,500 | 1,523,500 | 535,833 | 440,043 |
| 16.5 | 0.394 | 144,850 | 115,150 | 57,067 | 16,246 |
| 21 | 0.625 | 49,650 | 19,250 | 131,500 | 116,253 |
| 27 | 0.843 | 169,500 | 63,500 | 151,733 | 52,236 |

*Statistically significant difference

### Example 6

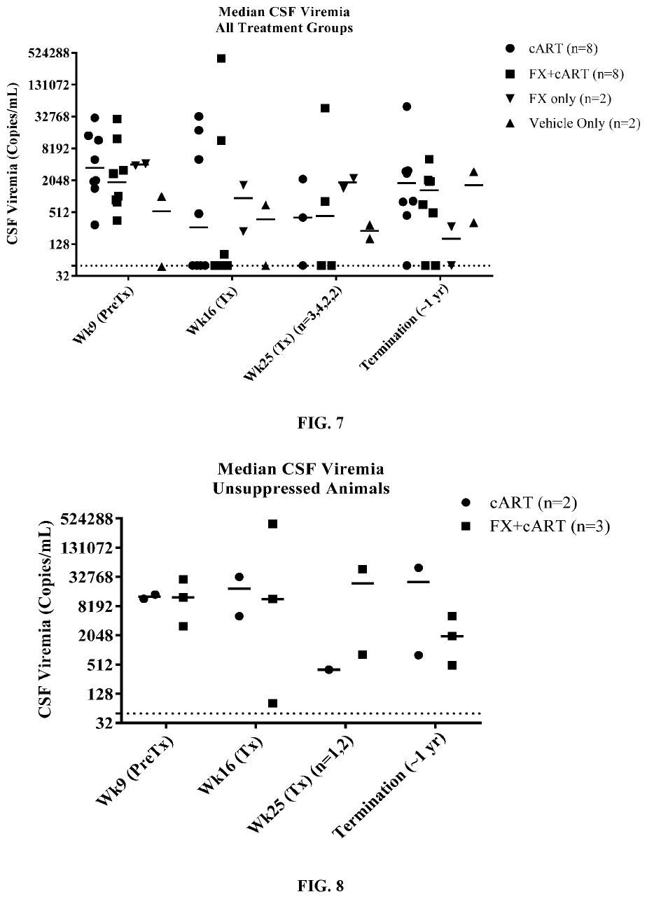

FX101 Treatment Alone and FX101 in Combination with cART Comparatively Reduces Viremia and Inflammation in the Central Nervous System Compartment (FIG. 7-10).

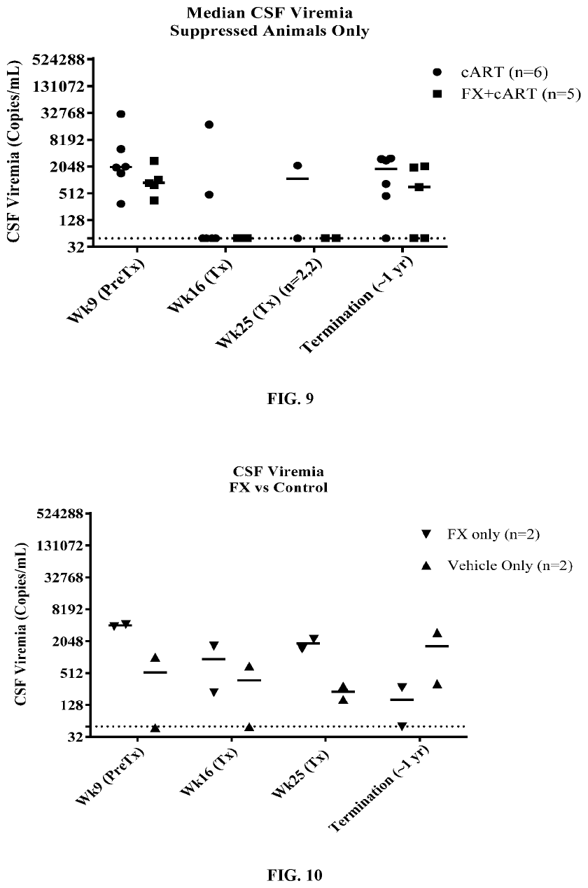

In the Central Nervous System (CNS), cerebrospinal fluid (CSF) viremia levels at week 16 (Wk16) post infection (6 weeks of therapy) were below detection (<50 RNA copies/mL) for all 5 of the 5 suppressed animals in the cotherapeutic FX+cART group, but for only 4 of the 6 suppressed animals in the cART group (FIG. 8 and FIG. 9). At Week 25 (Wk25), unfortunately, we had only two samples from each of these suppressed animal groups, with both samples below detection for the suppressed FX+cART group and one of two samples below detection for the suppressed cART group (Table III). At termination, the mean CSF viremia was 1698 RNA copies/mL for the suppressed cART group (n=6) and 959 RNA copies/mL for the suppressed FX+cART group (n=5). At termination, 2 of 5 suppressed FX+cART animals remained below detection while 1 of the 6 suppressed cART animals did. Comparative median CSF viremias were 2397 vs. 505 RNA copies/mL for cART (n=6) vs. FX+cART (n=5) groups at termination (FIG. 7 and FIG. 8), approaching statistical significance despite the small number of animals (p=0.125, Wilcoxin signed rank test, 2-tailed). These results support that FX+cART cotherapy suppresses viremia in the CSF compartment better than cART alone, providing benefit for this sanctuary site and recognized viral reservoir [A Carroll 2017].

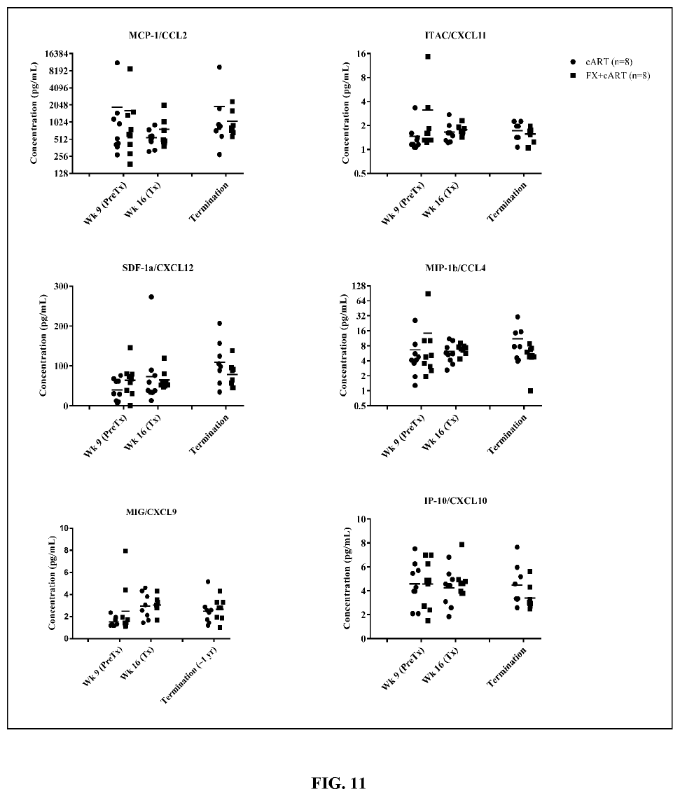

Surprisingly, CSF viremia levels were lower for the two (unsuppressed) monotherapeutic FX101-treated (“FX”) animals than for the control animals at termination (n=2 each group), despite the lack of any apparent plasma virus replication inhibition (FIG. 10 and Table IV). This may be related to penetration of Tier 2 neutralizing antibodies into the CNS compartment (which developed for both of these FX-treated animals) or to unique drug activities on viral pathologies in this compartmental viral reservoir. Interestingly, CSF viremia was lower in both control animals as compared with FX-treated animals through Wk25 study time points. Typically, microglia, macrophages and astrocytes are the infected cells in the CNS compartment. While the mean termination (˜1 year) CSF viremias for the “suppressed” cART group is closely equivalent to the “vehicle only” animals, CSF viremias for the FX-treated animals are also lower than for the “suppressed” cART group. Lower levels of MCP-1/CCL2 (monocyte chemoattractant protein-1), IP-10/CXCL10 (Interferon gamma-induced protein-10), ITAC/CXCL11 (Interferon-inducible T-cell alpha chemoattractant), SDF-1a/CXCL12 (stromal cell-derived factor-1 alpha) and MIP-1b/CCL4 (Macrophage inflammatory protein-1 beta), but not of MIG/CXCL9 (monokine induced by gamma interferon) in the CSF were measured for FX+cART-treated animals as compared with cART animals (FIG. 11), indicating comparative beneficial reductions of inflammation in the CNS compartment post treatment cessation [G V Raymond 2016].

TABLE III
Mean CSF Viremia for “Suppressed” Animals (RNA copies/mL)

| | cART Group (n = 6) | FX + cART Group (n = 5) |
|---|---|---|
| | Mean RNA copies/mL | Mean RNA copies/mL |
| Week 9 (PreTx) | 6907 | 1155 |
| Week 16 (Tx) | 3096 | <50 |
| Week 25 (Tx) | 1095 | <50 |
| (n = 2) | | |
| Termination | 1698 | 959 |

TABLE IV
Mean CSF Viremia FX versus Control (RNA copies/mL)

| | FX Only (n = 2) | Vehicle Only (n = 2) |
|---|---|---|
| | Mean RNA copies/mL | Mean RNA copies/mL |
| Week 9 (PreTx) | 4050 | 528 |
| Week 16 (Tx) | 940 | 372 |
| Week 25 (Tx) (n = 2) | 1850 | 226 |
| Termination | 161 | 1641 |

### Example 7

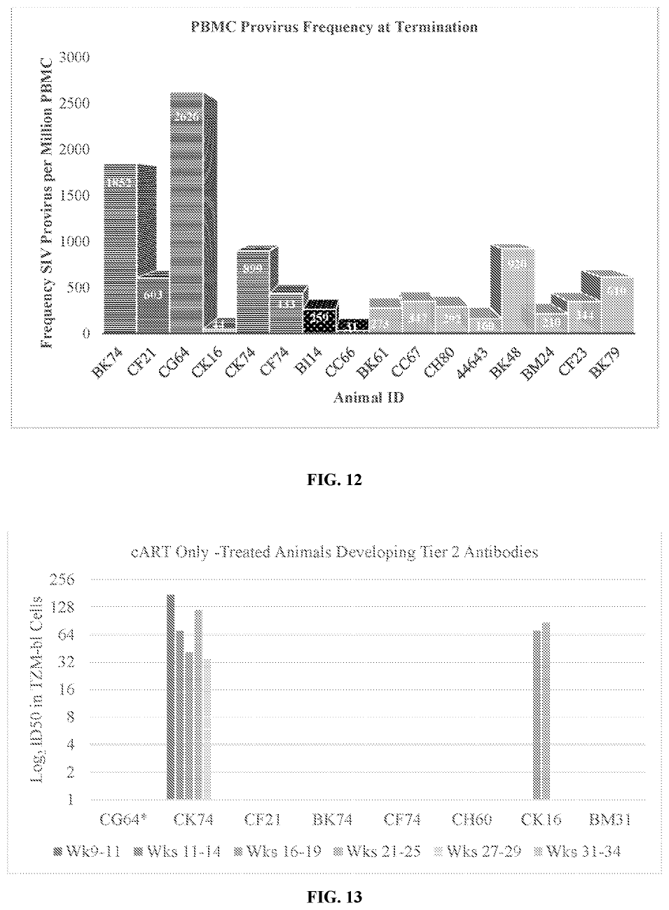

FX101 Treatment Alone, and/or FX101 in Combination with cART Resulted in Lower Frequencies of Provirus in Peripheral Blood Monocytic Cells (PBMC) as Compared with cART Treatment Alone (FIG. 12).

Genomic DNA was extracted from frozen (−80° C.), previously isolated (using ficoll separation) peripheral blood mononuclear cells (PBMCs) using Zymo or New England BioLabs DNA extraction kits as described by the manufacturer. DNA was further purified using DNA Clean and Concentrate kits from these same manufacturers. DNA purity was estimated using a ThermoFisher NanoDrop to meet a 260/280 ratio≥1.80 and a 260/230 ratio≥2.0. DNA was then diluted with Nuclease-free water to 2700 DNA copies/uL for optimizing Droplet Digital PCR (ddPCR) conditions. Samples were then digested with MscI restriction enzyme in CutSmart Buffer (New England BioLabs) for 60 minutes at 37° C., followed by enzyme deactivation for 20 minutes at 80° C., per the manufacturer's directions. Sufficient volumes per sample for 3-7 ddPCR replicate wells were processed for each DNA sample.

TABLE V
Primers and Probes for Measuring Proviral DNA

| Target | Forward primer | Reverse primer | Probe |
|---|---|---|---|
| SIV pol | GCAGGGATAGA | CTATGGTTTCTACT | 5′-/5Cy5/TT+TC+AG+GTG+GT |
| | GCACACCTTTG | GAATTTGCTTGTTC | GA+TT+CA/3IAbRQSp/-3′ |
| SIV env | CCTCAATAAAGC | GTTGTTGATGATTT | 5′-/5HEX/TG+C+ATTAC+TAT |
| | CTTGTGTAAAAT | TGTCAATCCC | +GA+GAT+GC/3IABKFQ/-3′ |
| | TATC | | |
| RPP30 | TCAGCATGGCGG | CGTGAGCCGCTGT | 5′-/6′FAM/TTCTGACCTGAAG |
| | TGTTTGCAGAC | CTCCACAAGTC | GCTCTGCGC/BHQ1aQ/-3′ |

Each digested DNA sample was added with primers, probes (Table V), Fluorescein (droplet counting dye), MasterMix and water to provide sample volumes of 27 μL (Table VII), which were mixed and loaded into each well of the Stilla Sapphire Chips (already prefilled with oil). The chips were then loaded into the Stilla Naica® Geode, which pressurizes the samples to generate oil droplets and then performs thermocycling. The thermocycling program was set at 95° C. for 10 minutes, followed by 45 cycles of 95° C. for 20 seconds with 55° C. annealing for 1 minute. Chips (“crystals”, which are the droplets in a 2-D space) were then imaged using the Naica® Prism3 to count the number of positive droplets for each target in each of the three detection channels. The data were interpreted using the Stilla Crystal Miner software, which compensates the signals and determines the copies/μL for each target. Double positive targets for SIV env and SIV pol were determined using the same software, which identifies droplets positive for both signals, distinguishing full length versus defective provirus. Frequencies for double positive droplets were then divided by the frequencies of cells, as determined by the copies/μL for Ribonuclease P Protein Subunit P30 (RPP30). The Naica® system has a limit of detection of 0.2 target copies/μL, with approximately 25,000 droplets per sample (“crystal”). Samples were pooled as needed to obtain a relative uncertainty in viral targets under 20% as determined by the Poisson distribution curve; the uncertainty in RPP30 cell copies was 1-2%.

TABLE VI
PCR Sample Processing

| Component | Target Concentration | |
|---|---|---|
| Each primer | 1 | μM |
| Each probe | 250 | nM |
| Fluorescein | 100 | nM |
| MasterMix (QuantaBio Perfecta | 1X | |
| Multiplex qPCR Supermix 5x) | | |
| DNA | 7.77 | ng/μL |

| | |
|---|---|
| Nuclease-free Water | To final volume of 27 μL |

Frequencies of total provirus in isolated PBMCs at study termination, as measured by double positive SIVenv and SIVpol sequences per total RPP30, is lower for the FX+cART cotherapy group than for the cART group (FIG. 12). Since none of the animals were virally suppressed at this time point, the frequency of provirus measured in PBMCs is a sum of both active virus replication and nonproductive latently-infected cells, including lymphocytes, monocytes and dendritic cells. While these results do not distinguish specific cell origins, PBMCs are typically comprised of ˜60% T lymphocytes, ˜15% monocytes/macrophages and ˜1% dendritic cells. Together with the associated lower viral setpoints, these results further support long term clinical benefit for FX101 adjunctive therapy with cART.

### Example 8

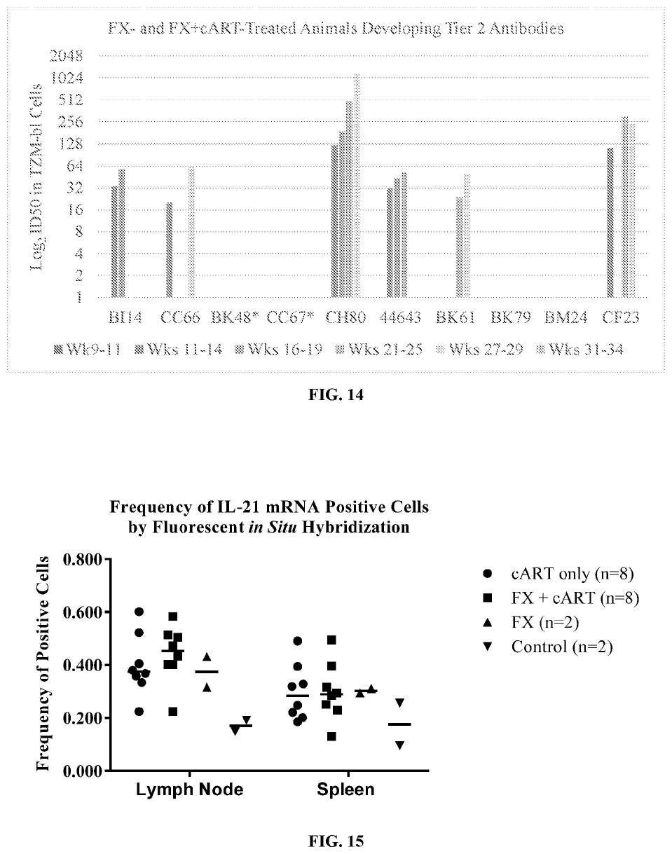

FX101 Treatment Alone, and/or FX101 in Combination with cART Resulted in Higher Tier 2 Antibody (Broadly Neutralizing) Production as Compared with cART Treatment Alone (FIG. 13-14).

Neutralizing antibodies titers from collected sera were measured. Over the study timeline, animals receiving FX101 monotherapy and/or in combination with cART produced more autologous Tier 2 (broadly neutralizing) antibodies by study termination (6 of 10, 60%) than animals receiving cART alone (2 of 8, 25%). Also, these titers are increasing over time for the FX-treated animals (FIG. 14). Importantly, male monkeys exhibit inferior innate and adaptive responses to lentivirus infections as compared with female monkeys, and to be slower to produce protective antibodies [I Tuero 2015; J George 2019; Kier Om 2020], this study included only male animals, emphasizing the remarkable findings of this study.

Some animals in this study produced very low levels of Tier 1 antibodies (BK48, CC67 and CG64) and could not be virally suppressed (“unsuppressed”) over the treatment period (FIG. 13-14). These responses are typical of rapid progressors. Of the animals producing Tier 1 antibodies, 6 of the 8 FX-treated animals (75%) produced Tier 2 antibodies while 2 of 7 cART (only)-treated animals produced Tier 2 antibodies (29%). Although BM24 (FX+cART group) did not produce Tier 2 antibodies by study termination, this animal-controlled viremia below the limit of detection for several months post treatment cessation, and therefore had lower levels of viral antigen than other animals, which may have delayed Tier 2 antibody development. Animal BK79 measured the lowest frequency of IL-21 production in lymph nodes of all other therapeutically treated animals, without wishing to be bound by theory, immunologically unique to this animal.

The chi-square test requires that % of the input counts be greater than 5 to represent an adequate sample population [Gilbert, STATISTICS p. 226]. The input counts for FX-treated animals (FIG. 14), excluding rapid progressors, defined by their inability to become virally suppressed (<50 RNA copies/mL) together with low to no production of Tier 1 antibodies: 6 (Tier 2), 2 (no Tier 2), cART (only)-treated animals (FIG. 12): 2 (Tier 2) and 5 (no Tier 2). As given, the chi-square test with Yates' correction (one-tailed for 1 degree of freedom) results in a P value of 0.10, which is within the 90% confidence interval. If a chi-square test is performed without a Yates' correction (one-tailed), the P value is 0.036 and this outcome is significant (alpha<0.05).

In HIV infection, between 10-30% of PLWH develop broadly neutralizing antibodies over time. This response generally evolves after 3 years [S Pallikkuth 2012]. The SIV/NHP study identifies a high proportion of FX101-treated animals producing Tier 2 antibodies as compared with animals receiving cART only at study termination, at equivalent times post infection and with similar viral loads over the treatment period. These results indicate that germinal centers are more functional in the FX101-treated animals, supporting better immunologic responses in this compartment, despite an ongoing virus infection that is known to impair production of autologous broadly neutralizing antibodies [E. Moysi 2018].

TZM-bl Assay for determining Tier 1 and Tier 2 Antibody Titers. Neutralizing antibody titers provide standardized assessments of the magnitude, breadth, kinetics and duration of vaccine-elicited neutralizing antibody responses in preclinical and clinical trials of candidate HIV and SIV vaccines using validated assays and standardized reference strains. Similar assays are used to assess the magnitude and breadth of neutralizing activity of serum samples and monoclonal antibodies from infected individuals. Antibody-mediated neutralization of HIV, SIV and SHIV is measured as a function of reductions in Tat-regulated Firefly luciferase (Luc) reporter gene expression after a single round of infection in TZM-bl cells. TZM-bl (also known as JC53BL-13) is a CXCR4-positive HeLa cell clone that was engineered to express CD4 and CCR5. The cells were further engineered to contain integrated reporter genes for firefly luciferase and Escherichia coli b-galactosidase under control of an HIV long-terminal repeat sequence. TZM-bl cells are permissive to infection by a wide variety of HIV, SIV and SHIV strains, including primary HIV isolates and molecularly cloned Env-pseudotyped viruses. Assay stocks of Env-pseudotyped viruses are produced in 293T/17 cells by co-transfection with an Env expression plasmid and a second plasmid expressing the entire HIV-1 genome except Env. Only the latter env-minus plasmid replicates in 293T/17 cells; this plasmid is packaged by the pseudovirions for delivery of the tat gene to TZM-bl cells. Thus, co-transfection generates pseudovirus particles that are infectious but are unable to produce infectious progeny virions for subsequent rounds of infection. Reporter gene expression is induced in trans by viral Tat protein soon after single cycle infection. Diethylethanolamine dextran is added to the medium to enhance infection and has been found to have no obvious effects on Neutralizing antibody activity. Luciferase activity is quantified as relative luminescence units (RLU) and is directly proportional to the number of infectious virus particles present in the initial inoculum over a wide range of values. Neutralization titers are the dilution at which RLU are reduced by 50% compared to virus control wells after subtraction of background RLUs. The assay is performed in 96-well plates for high throughput capacity and utilizes well-characterized reference strains for uniformity across studies [D C Montefiori 2004; J Mascola 2005; M Sarzotti-Kelsoe 2013].

### Example 9

Treatment of FX101 in Combination with cART (FX+cART) Results in a Higher Frequency of IL-21-Producing Cells in Lymph Nodes as Compared with cART Treatment Alone (FIG. 15).

The majority of lentivirus-infected cells reside in lymph tissues. Therefore, the impact of virus infection and beneficial use of FX101 therapy on this compartmental virus reservoir is critical to delineate. Immune responses of both the innate and adaptive pathways are centralized at lymph nodes where antigen presentation, cellular activation, infected cells and stowaway virus (on follicular dendritic cells, e.g.) can be difficult to therapeutically target. Lentivirus infection both directly and indirectly impairs multiple cellular immunologic functions.

The higher Tier 2 antibody productions indicated differences in germinal center capacities, so we performed (blinded) histochemical evaluation of axillary lymph node and spleen tissues collected at necropsy. Tissues were evaluated using fluorescent in situ hybridization (FISH) probes for each, IL-21 mRNA and SIV mRNA. Cells (stained with 4′,6-diamidino-2-phenylindole) were designated either positive or negative for each hybridization label. The frequency of positive cells was determined by dividing the positive signal by the total number of cells in the field of vision.

Evaluation of the results demonstrate that the FX+cART group measured higher frequencies of IL-21 positive cells in lymph nodes at study termination (˜1 year post infection) than the cART group (0.453 vs. 0.374, p=0.164 nonparametric Mann Whitney Test, Two-tailed n=8 per group) but not for spleen (0.2897 vs 0.2834, p=0.439). Animals had been off cART for ˜27 weeks at this time point, so this measure reflects a sustained immunologic reconstitution in the lymph nodes. The outlier in the FX+cART group (lowest point) measured the lowest CD4⁺ T cell count (e.g., 17% CD4⁺ T cells vs. −50% CD4⁺ T cells for other animals in the cohort at this time point), indicating that this animal may not have had sufficient T cells to generate IL-21 or may have had a genetic anomaly preventing that production, specific to that animal. Interestingly, frequencies of IL-21 mRNA positive cells in the FX-only treated animals were higher than for the control animals in both lymph nodes and spleen.

Immunohistochemistry and in situ hybridization of lymph node and spleen tissues collected at termination. Five μm formalin fixed paraffin embedded tissues sections were mounted on Superfrost Plus Microscope slides, baked overnight at 60° C. and passed through xylene, graded ethanol, and double distilled water to remove paraffin and rehydrate tissue sections. A microwave was used for heat induced epitope retrieval (HIER). Slides were boiled for 20 minutes in a Tris based solution, pH 9 (Vector Labs H-3301), containing 0.01% Tween20. Slides were briefly rinsed in hot, distilled water and transferred to a hot citrate-based solution, pH 6.0 (Vector Labs H-3300) where they were allowed to cool to room temperature. All slide manipulation from this point forward was done at room temperature. Once cool, slides were rinsed in tris buffered saline (TBS) and placed in a black, humidifying chamber where they were incubated with Background Punisher (Biocare Medical BP974H) for 10 minutes, washed with Tris Buffered Saline (TBS) containing 0.01% TritonX100 (TBS-TX100) for 5 minutes, followed by a quick rinse in TBS before being returned to the black chamber to be incubated with serum free protein block (Dako X0909) for 20 minutes. Rabbit polyclonal anti-IL21 primary antibody (Abcam, 1:500) was added to the slides for 60 minutes. Slides were then washed twice with TBS-TX100 and once with TBS. The labeling of the antibody for visualization was performed using the MACH3 AP kit (Biocare Medical M3R533). Both the MACH3 rabbit probe and polymer were incubated for 20 minutes with washes in between. Slides were incubated with Permanent Red substrate (Dako K0640) for 7 minutes and placed in TBS to halt the enzymatic reaction. After 3 additional washes, DAPI nuclear stain was added for 10 minutes. Slides were mounted using a homemade anti-quenching mounting media containing Mowiol (Calbiochem #475904) and 1,4-diazabicyclo[2.2.2]octane (Sigma #D2522) and allowed to dry overnight before imaging with a Zeiss Axio Scan.Z1 digital slide scanner.

In situ hybridization for the detection of simian immunodeficiency virus (SIV), was performed using the ViewRNA ISH Tissue Assay Core kit (Thermo, 19931) in combination with a custom probe set (Thermo, VF1-13908) designed to recognize sequences unique to gag, pol, and nef regions of SIV, thus identifying cells transcribing viral proteins. The manufacturer's protocol was adhered to for this assay. DAPI nuclear stain was added for 10 minutes. Slides were mounted using a homemade anti-quenching mounting media containing Mowiol (Calbiochem #475904) and 1,4-diazabicyclo[2.2.2]octane (Sigma #D2522) and allowed to dry overnight before imaging with a Zeiss Axio Scan.Z1 digital slide scanner.

Quantification of fluorescent immunohistochemistry and in situ hybridization. Stained slides were scanned with a Zeiss Axio Scan.Z1 digital slide scanner capturing up to three channels (405, 488, and 568). Whole slide images were analyzed using HALO image analysis software v3.1 (Indica Labs, Albuquerque, NM). Regions of interest were drawn around the tissue, excluding any tissue, stain, or scanning artifacts. The HighPlex FL module v3.1.0 was used to identify cells and determine positivity in the 488 and 568 channels. Thresholds were set manually by a pathologist using negative control slides. Negatives consisted of slides stained using a rabbit isotype control for IL-21 and a plant bacteria probe (DapB) for SIV. All regions of interest were subjected to the HALO algorithm. When quantifying SIV infected cells, areas with a dendritic pattern were excluded from the analysis. The numbers of positive cells were reported as a density (+cells/mm2) or a percentage (+cells/total cells). Comparison of treatment groups was assessed using a Mann-Whitney Test (2-tailed, nonparametric)

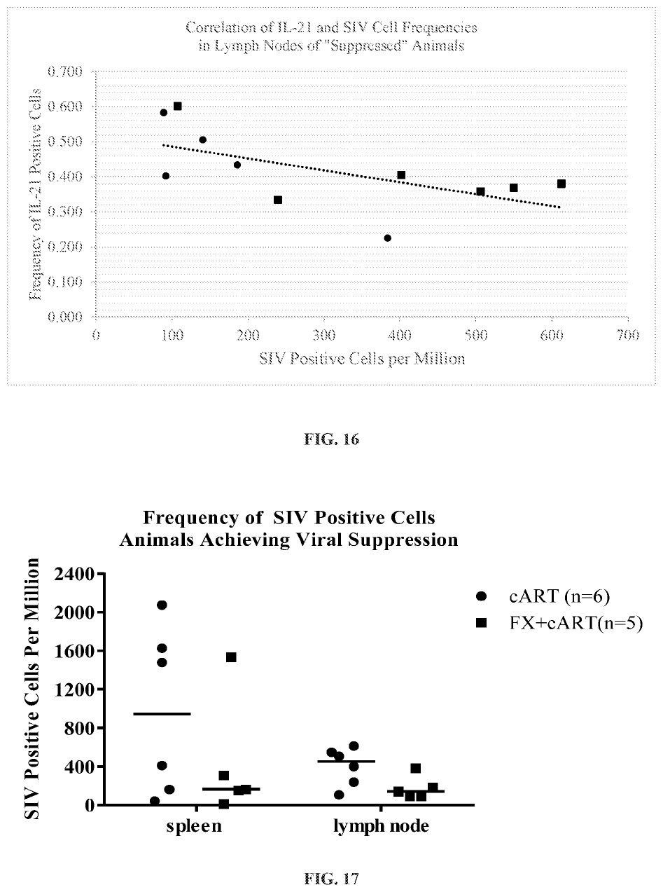

Beneficial value of IL-21 Production. We found that SIV mRNA positive cell frequencies inversely correlate with IL-21 mRNA positive cell frequencies in lymph nodes (FIG. 16 and Table VII) but not in spleen for animals achieving a period of plasma virus suppression (<50 RNA copies/mL). The significance of higher IL-21 mRNA production has been reported to facilitate HIV-specific cytotoxic (CD8⁺) T cell and NK cell functions and is associated with long term HIV control [H Elsaesser 2009; M Chevalier 2010; J S Yi 2010; L D Williams 2011; S. Buranapraditkun 2017] and is useful for a broad range of viral infections [H. Asao 2021]. IL-21 is also associated with development of plasma B cells and production of antiviral antibodies [B Schultz 2016; J Pušnik 2021]. Differences between IL-21 mRNA frequencies between treatment groups, as determined by fluorescent in situ hybridization probes in this study, may contribute to reduced viremia set points, lower SIV mRNA frequencies in tissue reservoirs (lymph node and spleen) [J Harper 2021] and Tier 2 antibody production responses. Importantly, peripheral blood circulating Tfh cells reflect this activity, and can be clinically monitored to assess this activity [S Boppana 2021]. Since lymphoid organs are the anatomical sites for generating antibodies, detecting higher frequencies of IL-21 positive cells in lymph nodes identifies IL-21-producing CD4⁺ T follicular helper cells (Tfh) as the cellular sources of this response. Tfh cells reside in both B cell follicles and germinal centers of lymphoid tissue, normally providing support to B cells toward developing antibodies and required to generate long-lived memory B cell responses [DA Vargas-Inchaustegui 2016; M Pauthner 2017]. Tfh cell quality and IL-21 expression have been identified as key factors distinguishing the development of autologous neutralizing antibodies, particularly in the development of Tier 2 neutralizing antibodies [C Havenar-Daughton 2016]. It is also known that HIV infection impairs Tfh function, and the development of vaccine-induced antibody responses [S Pallikkuth 2012]. In the absence of IL-21 signaling, germinal center B cells and long-lived plasma cells are not sustained, so that antibody responses are usually transient with the early collapse of germinal centers [M A Rasheed 2013]. As we continue to learn more regarding antibody production due to the SARS-CoV-2 pandemic, for instance, it is becoming clear that Tfh cells are critical for both the humoral immune response and the production of antibodies [R J Cox 2020]. The development of potent Tier 2 antibodies requires multiple rounds of a series of events involving refinement of memory B cell populations in long-lived germinal centers supported by IL-21-producing Tfh cells [R Nakagawa 2021]. Also, without wishing to be bound by theory, antigen-presenting cells (including dendritic cells) may play a role [J K Krishnaswamy 2017].

TABLE VII
Pearson Correlations of SIV mRNA and IL-21 mRNA
Frequencies for Virally Suppressed Animals

| | | P value | Significant? | | |
|---|---|---|---|---|---|
| | | (two- | (alpha = | | Pearson |
| | R² | tailed) | 0.05) | Equation | r |
| SIV mRNA with IL-21 | 0.3697 | 0.0472 | yes | Y = −1087x + 755 | −0.6080 |
| mRNA in Lymph Nodes | | | | | |
| SIV mRNA with IL-21 | 0.0599 | 0.2983 | no | N/A* | 0.2447 |
| mRNA in Spleen | | | | | |

*N/A means not applicable

Tfh cells also constitute a major part of the viral reservoir that persists during cART in addition to becoming dysfunctional during HIV/SIV infections [M Perreau 2013; R Banga 2016; Y Fukazawa 2015]. These cell subpopulations are considered a major focus in curative and remission strategies for HIV infection. As described herein, animals receiving FX101 alone or in combination with cART exhibited higher frequencies of IL-21 positive cells together with lower frequencies of SIV positive cells relative to comparative treatment groups, indicating Tfh cells are less infected and more highly functional with FX101 therapy. Furthermore, the production of IL-21 correlates with the successful production of autologous Tier 2 neutralizing antibodies in these animals.

The administration of IL-21 together with cART reduces inflammation, reduces immune activation, increases immune clearance functions, increases the expression of genes regulating antimicrobial immunity and reduce the viral reservoir in SIV-infected Rhesus macaques [L Micci 2015]. Therefore, the higher expression of IL-21 detected in this chronically-infected model indicates multiple benefits. Since IL-21 production is typically compromised during HIV/SIV infections, the differentiation of Th17 cells, maintenance of functional CD8⁺ T cells and antimicrobial immunity, and differentiation of memory B cells and antibody-secreting plasma cells are each negatively impacted. Morbidity and mortality in lentivirus infection can result from declines in CD4⁺ T lymphocytes and immune dysfunction. Ultimately, the inability to defend against opportunistic pathogenic infections is an AIDS-defining illness, with Cryptosporidium enteritis, cytomegalovirus pneumonia, Pneumocystis pneumonia, viral encephalitis, and lymphoma being prevalent causes of death. Therefore, the higher frequencies of IL-21-producing cells measured in the FX101-treated animals indicates a clinical benefit for treating lentivirus infections.

It is important to distinguish between spleen and lymph node tissues, recognizing that spleen (an organ) filters whole blood while lymph nodes (tissues) filter lymph. Although the spleen contains germinal centers, this organ is composed of multiple and unique cell types, including red blood cells in the red pulp with only one quarter of the tissue being white pulp, where B and T cells are concentrated. Additionally, the splenic red pulp regions include effector cells such as neutrophils, monocytes, dendritic cells, gamma delta T cells, CD8⁺ T cells and four major subtypes of splenic macrophages, with primarily innate immunity functions [S M Lewis 2019]. The distinction between Tfh-producing IL-21 cells and other innate immune cells in splenic tissues between treatment groups may be more difficult to determine. Furthermore, compartmental pathological differences in SIV-infected monkeys describe the depletion of Tfh cells expressing IL-21 early in infection in splenic tissues as compared with lymph node tissues [F Moukambi 2015] and a disproportionate higher CD8⁺ T cell population as compared with CD4⁺ T cells, which can also be sources of IL-21 production in lentivirus infection [Y Tian 2016].

Additionally, studies have shown a sex bias in Tfh function, with development of B cell immunity and protective antibody responses for male macaques being less successful than for female macaques [DA Vargas-Inchaustegui 2016]. All animals in this study were male, raising the significance of our study outcomes. Extending cART treatment beyond discontinuation of FX101 for an effective period of time is envisioned to further support a continuing reduction in the viral reservoir by protecting loss of CD4⁺ T cells, by supporting the immune response and by synergistically reducing virus populations in conjunction with IL-21 production activities, and by enhancing the potentials of broadly neutralizing antibodies.

### Example 10

Treatment of FX101 in Combination with cART Results in a Lower Frequency of SIV Positive Cells in Lymph Nodes and Spleen as Compared with cART Treatment Alone (FIG. 17-19).

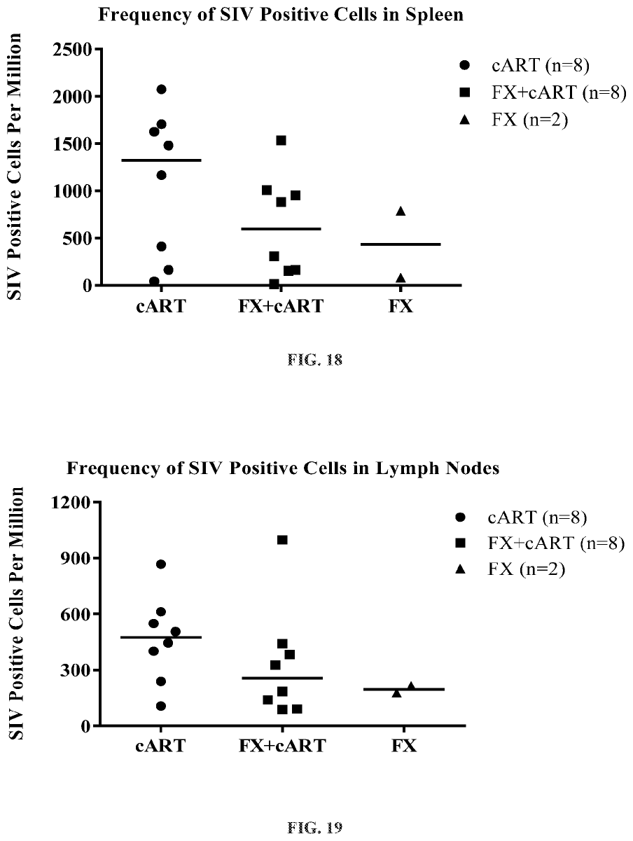

The frequency of SIV positive cells per million cells was determined using fluorescent in situ hybridization (FISH) as described in Example 8. The median frequencies of SIV-producing cells in both the spleen and lymph node tissues were found to be lower in the FX+cART group as compared with the cART group (Table VIII and FIG. 17) for animals achieving a period of plasma virus suppression (<50 RNA copies/mL), although the frequencies for all animals in the FX+cART group were lower in both spleen (FIG. 18) and lymph node tissues (FIG. 19) as compared with the cART group, regardless of whether they achieved a period of virus suppression (Table IX). Furthermore, the FX-only treatment group also exhibited very low frequencies of virus in both spleen and lymph nodes relative to the cART group (FIG. 18-19). Interestingly, the animal demonstrating the lowest IL-21 positive cells in each of the treatment groups has the corresponding highest SIV frequency in both tissues, consistent with the role of IL-21 production related to virus control in these tissues. Without wishing to be bound by theory, a genetic or immunologic abnormality can result in low IL-21 production, which has been observed to account for recurring tonsillitis in children [S Crotty 2019]. This animal may be a genetic outlier.

Tissue necropsies from long term (1-3 years) cART-suppressed SIV- and SHIV-infected Rhesus macaques identify lymph nodes as a primary source of continuing virus replication despite virus control (full suppression) in other anatomical sites [A M Cadena 2021]. The origin of this virus production appears to be directly from within the lymph node tissue provirus reservoir, emphasizing the unique challenges and benefits in therapeutically targeting this tissue.

Importantly, since macrophages can contribute to the viral reservoir and do not succumb to cytopathic effects of HIV infection [D W Williams 2016], their localization within the spleen and reduced frequency of SIV positive cells (by fluorescent in situ hybridization mRNA) in this organ for FX101-cotherapeutically treated animals indicates a reduction in the macrophage viral reservoir. For animals achieving a short period of suppression, this difference is approaching statistical significance in spleen, and does so for lymph node.

TABLE VIII
Frequencies of SIV Positive Cells in “Suppressed”
Animals determined by Fluorescent in Situ Hybridization
(FISH) at termination (~1 year)

| | Nonparametric Mann- | cART | FX + cART |
|---|---|---|---|
| | Whitney Test, | Group | Group |
| SIV Frequency per | 2- tailed | (n = 6) | (n = 5) |
| Million Cells | P value | Median | Median |
| spleen | 0.282 | 945 | 165 |
| lymph node | 0.053 | 454 | 140 |

TABLE IX
Frequencies of SIV Positive Cells in Lymph Nodes
and Spleen determined by Fluorescent in Situ
Hybridization (FISH) at termination (~1 year)

| | Nonparametric Mann- | cART | FX + cART |
|---|---|---|---|
| | Whitney Test, | Group | Group |
| SIV mRNA Frequency | 2- tailed | (n = 8) | (n = 8) |
| per Million Cells | P value | Median | Median |
| spleen | 0.195 | 1322 | 596 |
| lymph node | 0.130 | 476 | 257 |

### Example 11

No Observed Adverse Events Associated with FX101+cART Cotherapy as Measured by Multiple Safety Measures.

Safety was assessed by complete blood chemistries, clinical chemistries, flow cytometry, gross necropsies and histochemical tissue imaging. There were no apparent drug-related adverse events between treatment arms across these measures over the study timeline. However, since all the animals were SIV-infected, measures and observations outside of normal healthy expected ranges occurred. These measures and observations were not associated with a particular treatment group, as they occurred in each treatment arm. Measures outside normal ranges for complete blood counts and clinical chemistry such as lymphocyte and reticulocyte counts were observed in each treatment arm with equivalent distribution and are known to be associated with chronic SIV infection. Cotherapeutic treatment of FX101 alone or with cART at 3 mg/kg, 3 times weekly for up to 24 weeks is safe and well tolerated in this model, both as a monotherapy and as a cotherapy with cART, translationally relevant as a preclinical model for humans.

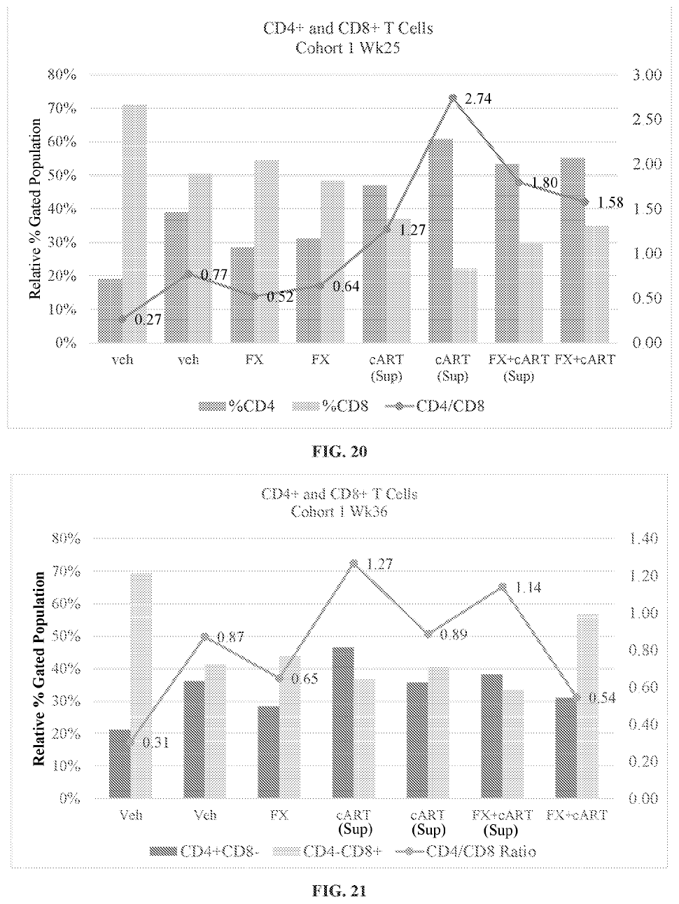

Additionally, no adverse effects on T cell subpopulations distributions were attributable to drug treatment as measured by flow cytometry. For Cohorts 1 and 2, for example (FIG. 20-23), Week 25 represents 15 weeks of therapy, commencing at Week 10. Differences between CD4⁺ and CD8⁺ subpopulations of CD3⁺ lymphocytes were a function of virus suppression (“Sup”), with suppressed animals having higher proportions of CD4⁺ subpopulations and unsuppressed animals having lower frequencies of CD4⁺ T cells, similar to control untreated (vehicle only) animals, with associated higher proportions of CD8⁺ T cells. Cotherapy with cART appears to have protected loss of CD4⁺ T cells.

Whole blood was collected at various times post infection to assess viral pathology and potential therapeutic treatment effects by flow cytometric analyses. One hundred microliters of whole blood were labeled using fluorescent antibodies for CD3⁺, CD4⁺ and CD8⁺ markers to identify CD4⁺ and CD8⁺ populations of the CD3⁺ T lymphocytes and relative changes in these T cell subpopulations. Blood was labeled using AF700 anti-CD3 (Biolegend cat #557917, clone SP34-2), APC-Cy7 anti-CD4 (Biolegend cat #317450, clone OKT4), and PE CD8beta (NIH NHP Reagent Resource, clone 255.1), incubated in the dark for 20 minutes, then lysed, washed and fixed using a BD FACS-Lyse Wash Assistant. The signals were then acquired on a BD LSR Fortessa™ following compensation and evaluated using FlowJo™ software. Lymphocyte populations were first gated, then CD3⁺ lymphocytes and finally each CD4⁺ and CD8⁺ subpopulation. The graphs (FIG. 20-23) indicate % of gated CD3⁺ lymphocytes.

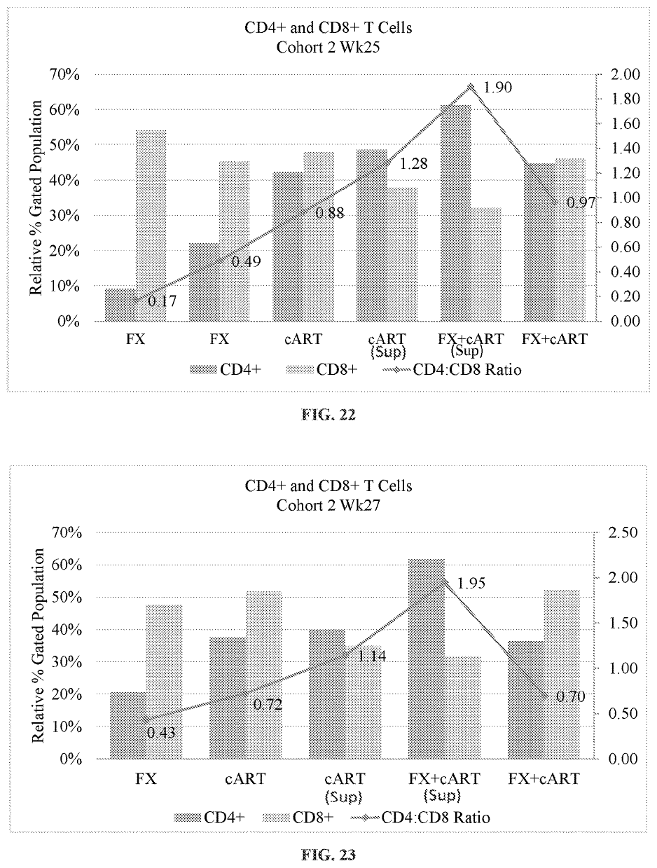

FIG. 20 and FIG. 22 illustrate the relative gated percentage of CD3⁺CD4⁺ T cells (left bar in each pair) after 15 weeks of therapeutic treatment (at 25 Weeks post infection) for Cohorts 1 and 2, including animals from all four treatment groups. These data show that cART cotherapy with FX101 (FX+cART) comparatively prevents loss of CD4⁺ T cells versus FX101 alone (FX), further benefiting animals achieving a period of virus suppression (“Sup”, <50 RNA copies/mL). At Week 36 (Wk36) post infection for Cohort 1 (11 weeks post cART therapeutic cessation) the CD4:CD8 ratios are highest for animals having achieved a period of suppression (FIG. 21). For Cohort 2, at Week 27 post infection (Wk27), animals had been off cART therapy for only 2 weeks, with the cotherapeutically suppressed (Sup) FX+cART animal comparatively preserving loss of CD4⁺ T cells with a correspondingly lower proportion of CD8⁺ T cells (right bar in each pair) as compared with other cART-treated animals (FIG. 23). The line with labeled values labeled (read from right axis) represents the ratio of CD4⁺/CD8⁺ T cells (CD4:CD8), which is greater than 1.0 (clinically beneficial) for most animals having achieved a period of virus suppression during therapy both during treatment and for multiple weeks post treatment cessation.

### SUMMARY

Both monotherapeutic treatment with FX101 and Cotherapeutic treatment of FX101 with cART was found to be safe and effective in this SIV/NHP model study. The translational benefit of FX101 cotherapy with cART provides long term (˜16 weeks post treatment cessation) reduction of virus production in tissue reservoirs (CNS, lymph node, spleen), increased frequency of IL-21 producing cells in lymph nodes and increased Tier 2 (broadly) neutralizing antibody productions, as well as long term reductions in the plasma viremia set point as compared with cART alone. FX101 therapy alone supports lower frequencies of virus-producing cells in lymphatic tissues and the development of autologous broadly neutralizing antibodies. The significance of these findings is that tissue reservoirs are recognized obstacles for therapeutic management of HIV infection. Additionally, broadly neutralizing antibodies are associated with long term control of HIV infection. In terms of the CNS, broadly neutralizing antibodies are considered one of the best immunotherapeutic strategies useful for controlling infection in this compartment [Kapoor 2020].

Continuing cART therapy for a limited period of time following cotherapy with FX101 may further reduce the viral burden by minimizing immunologic dysfunction and T cell loss inherent with the onset of active virus replication. Furthermore, in the presence of Tier 2 antibody production, a limited period of cART therapy may help to further reduce the latent reservoir [Caskey 2020] as a therapeutic strategy toward long term remission and HIV cure.

Owing to the highly conserved residues (usually cysteine and histidine) required to coordinate zinc, which continue to be identified in other current and emerging viruses, together with the viral protein-host protein interactions which subvert host cell responses, the results of this study in chronically SIV-infected Rhesus macaques support long term antiviral activities and a good safety profile for treatment of virus infections of multiple virus families with compounds of this invention. Viruses known to comprise zinc domains in viral proteins include, but are not limited to, other Retroviruses, Bunyaviruses, Arenaviruses, Togaviruses, Paramyxoviruses, Flaviviruses, Hepadnaviruses and Coronaviruses. Compounds of this invention can be administered at an effective amount, using a suitable formulation and dosing route to treat virus infections wherein one or more viral proteins comprise zinc domains.

### Example 12

1. Abbehausen, C., Zinc finger domains as therapeutic targets for metal-based compounds—an update. Metallomics, 2019. 11(1): p. 15-28.
2. Asao, H., Interleukin-21 in Viral Infections. Int J Mol Sci, 2021. 22(17).
3. Audsley, M. D. and G. W. Moseley, Paramyxovirus evasion of innate immunity: Diverse strategies for common targets. World J Virol, 2013. 2(2): p. 57-70.
4. Banga, R., et al., PD-1(+) and follicular helper T cells are responsible for persistent HIV-1 transcription in treated aviremic individuals. Nat Med, 2016. 22(7): p. 754-61.
5. Barouch, D. H., et al., Therapeutic efficacy of potent neutralizing HIV-1-specific monoclonal antibodies in SHIV-infected rhesus monkeys. Nature, 2013. 503(7475): p. 224-8.
6. Boppana, S., et al., SARS-CoV-2-specific circulating T follicular helper cells correlate with neutralizing antibodies and increase during early convalescence. PLoS Pathog, 2021. 17(7): p. e1009761.
7. Borducchi, E. N., et al., Ad26/MVA therapeutic vaccination with TLR7 stimulation in SIV-infected rhesus monkeys. Nature, 2016. 540(7632): p. 284-287.
8. Buranapraditkun, S., et al., Preservation of Peripheral T Follicular Helper Cell Function in HIV Controllers. J Virol, 2017. 91(14).
9. Cadena, A. M., et al., Persistence of viral RNA in lymph nodes in ART-suppressed SIV/SHIV-infected Rhesus Macaques. Nat Commun, 2021. 12(1): p. 1474.
10. Carroll, A. and B. Brew, HIV-associated neurocognitive disorders: recent advances in pathogenesis, biomarkers, and treatment. F1000Res, 2017. 6: p. 312.
11. Caskey, M., Broadly neutralizing antibodies for the treatment and prevention of HIV infection. Curr Opin HIV AIDS, 2020. 15(1): p. 49-55.
12. Chahroudi, A. and G. Silvestri, HIV and Tfh Cells: Circulating New Ideas to Identify and Protect. Immunity, 2016. 44(1): p. 16-18.
13. Chevalier, M. F., et al., HIV-1-specific interleukin-21+ CD4+ T cell responses contribute to durable viral control through the modulation of HIV-specific CD8+ T cell function. J Virol, 2011. 85(2): p. 733-41.
14. Cox, R. J. and K. A. Brokstad, Not just antibodies: B cells and T cells mediate immunity to COVID-19. Nat Rev Immunol, 2020. 20(10): p. 581-582.
15. Crotty, S., T Follicular Helper Cell Biology: A Decade of Discovery and Diseases. Immunity, 2019. 50(5): p. 1132-1148.
16. Del Prete, G. Q., et al., Short Communication: Comparative Evaluation of Coformulated Injectable Combination Antiretroviral Therapy Regimens in Simian Immunodeficiency Virus-Infected Rhesus Macaques. AIDS Res Hum Retroviruses, 2016. 32(2): p. 163-8.
17. Delgado, C. P., et al., In silico studies of M(pro) and PL(pro) from SARS-CoV-2 and a new class of cephalosporin drugs containing 1,2,4-thiadiazole. Struct Chem, 2022. 33(6): p. 2205-2220.
18. Dunham, R. M., et al., Preclinical evaluation of HIV eradication strategies in the simian immunodeficiency virus-infected rhesus macaque: a pilot study testing inhibition of indoleamine 2,3-dioxygenase. AIDS Res Hum Retroviruses, 2013. 29(2): p. 207-14.
19. Elsaesser, H., K. Sauer, and D. G. Brooks, IL-21 is required to control chronic viral infection. Science, 2009. 324(5934): p. 1569-72.
20. Esposito, S., et al., Host and Viral Zinc-Finger Proteins in COVID-19. Int J Mol Sci, 2022. 23(7).
21. Estrada, D. F., et al., The Structure of the Hantavirus Zinc Finger Domain is Conserved and Represents the Only Natively Folded Region of the Gn Cytoplasmic Tail. Front Microbiol, 2011. 2: p. 251.
22. Estrada, D. F. and R. N. De Guzman, Structural characterization of the Crimean-Congo hemorrhagic fever virus Gn tail provides insight into virus assembly. J Biol Chem, 2011. 286(24): p. 21678-86.
23. Fukazawa, Y., et al., B cell follicle sanctuary permits persistent productive simian immunodeficiency virus infection in elite controllers. Nat Med, 2015. 21(2): p. 132-9.
24. Gao, Y., et al., Multiple roles of the non-structural protein 3 (nsP3) alphavirus unique domain (AUD) during Chikungunya virus genome replication and transcription. PLoS Pathog, 2019. 15(1): p. e1007239.
25. Garcia, C. C. and E. B. Damonte, Zn finger containing proteins as targets for the control of viral infections. Infect Disord Drug Targets, 2007. 7(3): p. 204-12.
26. George, J., et al., Gender differences in innate responses and gene expression profiles in memory CD4 T cells are apparent very early during acute simian immunodeficiency virus infection. PLoS One, 2019. 14(9): p. e0221159.
27. Harper, J., et al., IL-21 and IFNalpha therapy rescues terminally differentiated NK cells and limits SIV reservoir in ART-treated macaques. Nat Commun, 2021. 12(1): p. 2866.
28. Hatziioannou, T. and D. T. Evans, Animal models for HIV/AIDS research. Nat Rev Microbiol, 2012. 10(12): p. 852-67.
29. Havenar-Daughton, C., et al., Direct Probing of Germinal Center Responses Reveals Immunological Features and Bottlenecks for Neutralizing Antibody Responses to HIV Env Trimer. Cell Rep, 2016. 17(9): p. 2195-2209.
30. Hokello, J., et al., Human Immunodeficiency Virus Type-1 (HIV-1) Transcriptional Regulation, Latency and Therapy in the Central Nervous System. Vaccines (Basel), 2021. 9(11).
31. Kapoor, A. and C. S. Tan, Immunotherapeutics to Treat HIV in the Central Nervous System. Curr HIV/AIDS Rep, 2020. 17(5): p. 499-506.
32. Kar, M., et al., Zinc Chelation Specifically Inhibits Early Stages of Dengue Virus Replication by Activation of NF-kappaB and Induction of Antiviral Response in Epithelial Cells. Front Immunol, 2019. 10: p. 2347.
33. Keiffer, T. R., et al., Interactions of the Nipah Virus P, V, and W Proteins across the STAT Family of Transcription Factors. mSphere, 2020. 5(6).
34. Kleinman, A. J., I. Pandrea, and C. Apetrei, So Pathogenic or So What?—A Brief Overview of SIV Pathogenesis with an Emphasis on Cure Research. Viruses, 2022. 14(1).
35. Klingler, J., et al., How HIV-1 Gag Manipulates Its Host Cell Proteins: A Focus on Interactors of the Nucleocapsid Domain. Viruses, 2020. 12(8).
36. Krishnaswamy, J. K., et al., Migratory CD11b(+) conventional dendritic cells induce T follicular helper cell-dependent antibody responses. Sci Immunol, 2017. 2(18).
37. Lewis, S. M., A. Williams, and S. C. Eisenbarth, Structure and function of the immune system in the spleen. Sci Immunol, 2019. 4(33).
38. Liu, P. T., et al., Origin of rebound virus in chronically SIV-infected Rhesus monkeys following treatment discontinuation. Nat Commun, 2020. 11(1): p. 5412.
39. Micci, L., et al., Interleukin-21 combined with ART reduces inflammation and viral reservoir in SIV-infected macaques. J Clin Invest, 2015. 125(12): p. 4497-513.
40. Miller-Novak, L. K., et al., Analysis of Complement-Mediated Lysis of Simian Immunodeficiency Virus (SIV) and SIV-Infected Cells Reveals Sex Differences in Vaccine-Induced Immune Responses in Rhesus Macaques. J Virol, 2018. 92(19).
41. Monette, A., et al., Pan-retroviral Nucleocapsid-Mediated Phase Separation Regulates Genomic RNA Positioning and Trafficking. Cell Rep, 2020. 31(3): p. 107520.
42. Mouhand, A., et al., Overview of the Nucleic-Acid Binding Properties of the HIV-1 Nucleocapsid Protein in Its Different Maturation States. Viruses, 2020. 12(10).
43. Moukambi, F., et al., Early Loss of Splenic Tfh Cells in SIV-Infected Rhesus Macaques. PLoS Pathog, 2015. 11(12): p. e1005287.
44. Moysi, E., C. Petrovas, and R. A. Koup, The role of follicular helper CD4 T cells in the development of HIV-1 specific broadly neutralizing antibody responses. Retrovirology, 2018. 15(1): p. 54.
45. Nakagawa, R., et al., Permissive selection followed by affinity-based proliferation of GC light zone B cells dictates cell fate and ensures clonal breadth. Proc Natl Acad Sci USA, 2021. 118(2).
46. Okoye, A. A., et al., Early antiretroviral therapy limits SIV reservoir establishment to delay or prevent post-treatment viral rebound. Nat Med, 2018. 24(9): p. 1430-1440.
47. Om. K., et al., Adjuvanted HIV-1 vaccine promotes antibody-dependent phagocytic responses and protects against heterologous SHIV challenge. PLoS Pathog, 2020. 16(9): p. e1008764.
48. Pallikkuth, S., A. Parmigiani, and S. Pahwa, Role of IL-21 and IL-21 receptor on B cells in HIV infection. Crit Rev Immunol, 2012. 32(2): p. 173-95.
49. Pallikkuth, S., et al., Impaired peripheral blood T-follicular helper cell function in HIV-infected nonresponders to the 2009 H1N1/09 vaccine. Blood, 2012. 120(5): p. 985-93.
50. Pauthner, M., et al., Elicitation of Robust Tier 2 Neutralizing Antibody Responses in Nonhuman Primates by HIV Envelope Trimer Immunization Using Optimized Approaches. Immunity, 2017. 46(6): p. 1073-1088 e6.
51. Perreau. M., et al., Follicular helper T cells serve as the major CD4 T cell compartment for HIV-1 infection, replication, and production. J Exp Med, 2013. 210(1): p. 143-56.
52. Policicchio, B. B., I. Pandrea, and C. Apetrei, Animal Models for HIV Cure Research. Front Immunol, 2016. 7: p. 12.
53. Pusnik, J., et al., Production of HIV-1 Env-specific antibodies mediating innate immune functions depends on cognate IL-21-secreting CD4+ T cells. J Virol, 2021.
54. Ramakrishnan, D., et al., Hepatitis B Virus X Protein Function Requires Zinc Binding. J Virol, 2019. 93(16).
55. Rasheed, M. A., et al., Interleukin-21 is a critical cytokine for the generation of virus-specific long-lived plasma cells. J Virol, 2013. 87(13): p. 7737-46.
56. Raymond, G. V., et al., Elevated cerebral spinal fluid biomarkers in children with mucopolysaccharidosis I-H. Sci Rep, 2016. 6: p. 38305.
57. Sanchez-Aparicio, M. T., et al., Paramyxovirus V Proteins Interact with the RIG-I/TRIM25 Regulatory Complex and Inhibit RIG-I Signaling. J Virol, 2018. 92(6).
58. Scholz, E. M. B. and A. D. M. Kashuba, The Lymph Node Reservoir: Physiology, HIV Infection, and Antiretroviral Therapy. Clin Pharmacol Ther, 2021. 109(4): p. 918-927.
59. Schultz, B. T., et al., Circulating HIV-Specific Interleukin-21(+)CD4(+) T Cells Represent Peripheral Tfh Cells with Antigen-Dependent Helper Functions. Immunity, 2016. 44(1): p. 167-178.
60. Tarasova, O., et al., Data and Text Mining Help Identify Key Proteins Involved in the Molecular Mechanisms Shared by SARS-CoV-2 and HIV-1. Molecules, 2020. 25(12).
61. Terrade, G., et al., Interests of the Non-Human Primate Models for HIV Cure Research. Vaccines (Basel), 2021. 9(9).
62. Tian, Y. and A. J. Zajac, IL-21 and T Cell Differentiation: Consider the Context. Trends Immunol, 2016. 37(8): p. 557-568.
63. Tuero, I., et al., Mucosal B Cells Are Associated with Delayed SIV Acquisition in Vaccinated Female but Not Male Rhesus Macaques Following SIVmac251 Rectal Challenge. PLoS Pathog, 2015. 11(8): p. e1005101.
64. Twizerimana, A. P., et al., Cell Type-Dependent Escape of Capsid Inhibitors by Simian Immunodeficiency Virus SIVcpz. J Virol, 2020. 94(23).
65. Uchida, S. et al., Possible role of the Nipah virus V protein in the regulation of the interferon beta induction by interacting with UBX domain-containing protein1. Sci Rep 2018. 8, 7682.
66. Van Rompay, K. K. A., Tackling HIV and AIDS: contributions by non-human primate models. Lab Anim (NY), 2017. 46(6): p. 259-270.
67. Vargas-Inchaustegui, D. A., et al., Vaccine Induction of Lymph Node-Resident Simian Immunodeficiency Virus Env-Specific T Follicular Helper Cells in Rhesus Macaques. J Immunol, 2016. 196(4): p. 1700-10.
68. Wang, G., et al., Physiological Concentration of H(2)O(2) Supports Dopamine Neuronal Survival via Activation of Nrf2 Signaling in Glial Cells. Cell Mol Neurobiol, 2021. 41(1): p. 163-171.
69. Wang Y, et al., The Zinc Content of HIV-1 NCp7 Affects Its Selectivity for Packaging Signal and Affinity for Stem-Loop 3. Viruses. 2021 Sep. 24:13(10):1922.
70. Whitney, J. B., et al., Rapid seeding of the viral reservoir prior to SIV viraemia in rhesus monkeys. Nature, 2014. 512(7512): p. 74-7.
71. Williams, D. W., et al., Splenic Damage during SIV Infection: Role of T-Cell Depletion and Macrophage Polarization and Infection. Am J Pathol, 2016. 186(8): p. 2068-2087.
72. Williams, L. D., et al., Interleukin-21-producing HIV-1-specific CD8 T cells are preferentially seen in elite controllers. J Virol, 2011. 85(5): p. 2316-24.
73. Yap, T. L., et al., Crystal structure of the dengue virus RNA-dependent RNA polymerase catalytic domain at 1.85-angstrom resolution. J Virol, 2007. 81(9): p. 4753-65.
74. Yi, J. S., M. A. Cox, and A. J. Zajac, Interleukin-21: a multifunctional regulator of immunity to infections. Microbes Infect, 2010. 12(14-15): p. 1111-9.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating or preventing a viral infection, or alleviating a symptom thereof, the method comprising administering to a subject a compound according to Formula (1a) or Formula (1b) 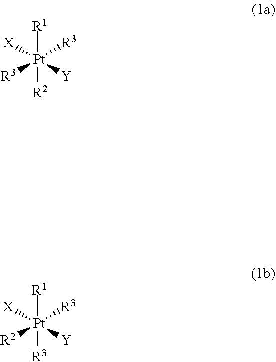
wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻;
wherein R¹ is NO₂, COOH, COOR², COR, OH or SO₃H;
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, alkoxycarbonyl, heteroaryloxycarbonyl, heterocycloalkoxy, or heterocycloalkoxycarbonyl;
wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

2. The method of claim 1, wherein X and Y are Cl, R¹ is NO₂, R² is Cl, and R³ is NH₃.

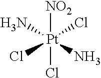

3. The method of claim 1, wherein the orientation of X and Y are trans to one another.

4. The method of claim 1, wherein the compound is selected from the group consisting of a compound according to Formula (1a) or Formula (1b) 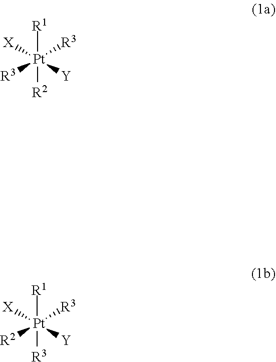
wherein X and Y are independently, nucleophilic exchangeable leaving groups,
wherein R¹ is NO₂ or OH,
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)₂, phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl,
wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

5. The method of claim 1, wherein the virus comprises a lentivirus.

6. The method of claim 5, wherein the lentivirus comprises HIV, SIV, SHIV, or FIV.

7. The method of claim 1, wherein the virus is not HIV, SIV, SHIV, or FIV.

8. The method of claim 1, wherein the virus comprises a Retrovirus, Coronavirus, Paramyxovirus, Togavirus, Flavivirus, Bunyavirus, or a Hepadnavirus.

9. The method of claim 8, wherein the virus comprises an Oncoretrovirus, Human T-lymphotropic virus (HTLV), Feline lymphotropic virus (FeLV), Spumavirus, a Nidovirus, Severe Respiratory Syndrome Corona Virus 2 (SARS-CoV-2), Middle East Respiratory Syndrome (MERS-CoV), Severe Respiratory Syndrome Corona Virus 1 (SARS-CoV-1), a Henipavirus, Measles virus (MeV), Nipah virus (NiV), Mumps virus (MuV), Sendai virus (SeV), Parainfluenza virus 5 (PIV-5), Human parainfluenza virus (HPIV); Hendra virus (HeV), Newcastle Disease virus (NDV), an Alphavirus, Chikungunya virus (CHIKV), Sindbis virus (SINV), Dengue virus-1, Dengue virus-2, Dengue virus-3, Dengue Virus-4, West Nile virus (WNV), Japanese encephalitis virus (JEV), Zika virus, Yellow Fever Virus (YFV), a Nairovirus, Crimean Congo Hemorrhagic Fever (CCHF) virus, a Hantavirus, an Orthobunyavirus, an Arenavirus, or Hepatitis B virus (HBV).

10. The method of claim 1, further comprising administering to the subject at least one additional antiviral therapy.

11. The method of claim 1, wherein the subject has previously been treated with at least one additional antiviral therapy.

12. The method of claim 10 or 11, wherein the antiviral therapy comprises an antiretroviral therapy.

13. The method of claim 10 or 11, wherein the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

14. The method of claim 13, wherein the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1 or any combination thereof.

15. The method of claim 10 or 11, wherein the antiviral therapy comprises a broadly neutralizing antibody.

16. The method of claim 15, wherein the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

17. The method of claim 10 or 11, wherein the antiviral therapy comprises combination antiretroviral therapy (cART).

18. The method of claim 1, wherein treating or preventing a viral infection is indicated by reduced lentivirus levels in lymphatic tissue, reduced lentivirus in cerebrospinal fluid, increased IL-21 production in lymphatic tissue, development of broadly neutralizing antibodies, reduced viral setpoint, reduced frequency of provirus in peripheral blood mononuclear cells, or any combination thereof.

19. A method of treating or preventing a viral infection, or alleviating a symptom thereof, the method comprising administering to a subject a first agent and a second agent, wherein the first agent comprises an antiviral therapy, and wherein the second agent comprises a compound according to Formula (1a) or Formula (1b) 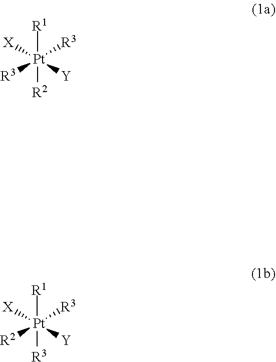
wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻;
wherein R¹ is NO₂, COOH, COOR², OR, COR, OH or SO₃H;
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (COR²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OSO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

20. The method of claim 19, wherein X and Y are Cl, R¹ is NO₂, R² is Cl, and R³ is NH₃.

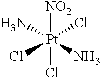

21. The method of claim 19, wherein the orientation of X and Y are trans to one another.

22. The method of claim 19, wherein the compound is selected from the group consisting of a compound according to Formula (1a) or Formula (1b), 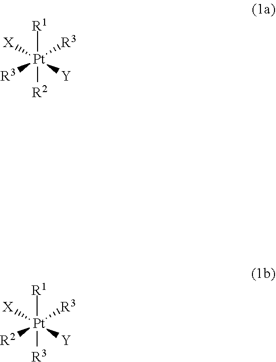
wherein X and Y are independently, nucleophilic exchangeable leaving groups;
wherein R¹ is NO₂ or OH,
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)₂, phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

23. The method of claim 19, wherein the first agent and the second agent are administered simultaneously or sequentially.

24. The method of claim 19, wherein the virus comprises a lentivirus.

25. The method of claim 24, wherein the lentivirus comprises HIV, SIV, SHIV, or FIV.

26. The method of claim 19, wherein the virus is not HIV, SIV, SHIV, or FIV.

27. The method of claim 19, wherein the virus comprises a Retrovirus, Coronavirus, Paramyxovirus, Togavirus, Flavivirus, Bunyavirus, or a Hepadnavirus.

28. The method of claim 27, wherein the an Oncoretrovirus, Human T-lymphotropic virus (HTLV), Feline lymphotropic virus (FeLV), Spumavirus, a Nidovirus, Severe Respiratory Syndrome Corona Virus 2 (SARS-CoV-2), Middle East Respiratory Syndrome (MERS-CoV), Severe Respiratory Syndrome Corona Virus 1 (SARS-CoV-1), a Henipavirus, Measles virus (MeV), Nipah virus (NiV), Mumps virus (MuV), Sendai virus (SeV), Parainfluenza virus 5 (PIV-5), Human parainfluenza virus (HPIV); Hendra virus (HeV), Newcastle Disease virus (NDV), an Alphavirus, Chikungunya virus (CHIKV), Sindbis virus (SINV), Dengue virus-1, Dengue virus-2, Dengue virus-3, Dengue Virus-4, West Nile virus (WNV), Japanese encephalitis virus (JEV), Zika virus, Yellow Fever Virus (YFV), a Nairovirus, Crimean Congo Hemorrhagic Fever (CCHF) virus, a Hantavirus, an Orthobunyavirus, an Arenavirus, or Hepatitis B virus (HBV).

29. The method of claim 19, wherein the antiretroviral therapy comprises combination antiretroviral therapy (cART).

30. The method of claim 19, wherein the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

31. The method of claim 19, wherein the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1, or any combination thereof.

32. The method of claim 19, wherein the antiviral therapy comprises a broadly neutralizing antibody.

33. The method of claim 32, wherein the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.

34. The method of claim 19, wherein treating or preventing a viral infection is indicated by reduced lentivirus levels in lymphatic tissue, reduced lentivirus in cerebrospinal fluid, increased IL-21 production in lymphatic tissue, development of broadly neutralizing antibodies, reduced viral setpoint, reduced frequency of provirus in peripheral blood mononuclear cells, or any combination thereof.

35. An antiviral composition comprising a first agent and a second agent, wherein the first agent comprises an antiviral therapy, and wherein the second agent comprises a compound according to Formula (1a) or Formula (1b) 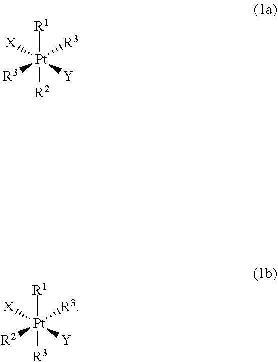
wherein X and Y are, independently, F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², acetate (CH₃COO⁻), acetoxy (carboxylate (CO₂R²)⁻, sulfate (SO₄)², phosphate (HPO₄)²⁻, selenate (SeO₄)²⁻, or silicate (SiO₄)²⁻;
wherein R¹ is NO₂, COOH, COOR², OR, COR, OH or SO₃H;
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OBO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alky carbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
wherein R³ is NH₃, NH₂R², NH(R²)₂, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, sulfide, sulfoxide, or thiourea derivative, or a pharmaceutically acceptable salt, crystal, co-crystal, prodrug, or solvate thereof, or any combination thereof.

36. The antiviral composition of claim 35, wherein X and Y are Cl, R¹ is NO₂, R² is Cl, and R³ is NH₃.

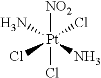

37. The antiviral composition of claim 35, wherein the orientation of X and Y are trans to one another.

38. The antiviral composition of claim 35, wherein the compound is selected from the group consisting of a compound according to Formula (1a) or Formula (1b), 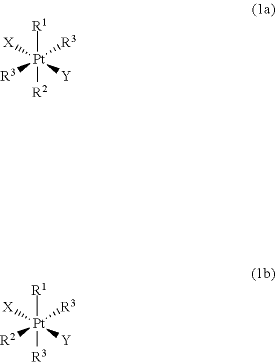
wherein X and Y are independently, nucleophilic exchangeable leaving groups;
wherein R¹ is NO₂ or OH;
wherein R² is F, Cl, Br, I, CN, SCN, NCS, NO₂, ONO, OHSO₃, OH₂PO₃, OHSO₂, SO₃H, OH, OR², OS(CH₃)₂, OCOR², OCOOR², OSO₂CH₃, OS(CH₃)₂, SH, SR², SC(NH₂)₂, S₂CN(R²)₂, OSiO₃, OSO₂H, OHSeO₂, NHCOH, NH₂CHO, NH₂CH₂OH, NH₂C(OH)₃, NH₂CH(OH)₂, NHCOR², carboxylate (CO₂R²)⁻, sulfate (SO₄)², phosphate (HPO₄)²⁻, selenate (SeO₄)²⁻, or silicate (SiO₄)², an alkyl, alkoxy, cycloalkyl, cycloalkoxy, aryl, aryloxy, alkycarbonyl, alkoxycarbonyl, cycloalkylcarbonyl, dimethylsulfoxide, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl, any of which can be optionally substituted with F, Cl, Br, I, COOH, OH, NO₂, NH₂, HSO₃, OH₂PO₃, OBO₂, OHSiO₃, OHSeO₂, N-alkyl, alkyl, alkoxy, cycloalkyl, cycloalkoxy, aryl, aryloxy, alkycarbonyl, alkoxycarbonyl, cycloalkylcarbonyl, heteroalkyl, heterocycloalkyl, heterocycloalkylcarbonyl, heteroaryl, arylcarbonyl, heteroarylcarbonyl, aryloxycarbonyl, heteroaryloxycarbonyl, heterocycloalkoxy, or heterocycloalkoxycarbonyl;
wherein R³ is NH₃, NH₂R², NH(R²)₃, N(R²)₃, NH₂COR², NH₂COH, NH₂CHO, NH₂CH₂OH, NH₂C(OH)₃, NH₂CH(OH)₂, NCR, OCH₃, OR², an amine, amidine, nitrile, iminoether, N-heterocycle, pyrimidine, pyridine or functionalized pyridine, imidazothiazole, xanthine, aliphatic amine, S-heterocycle, sulfide, sulfoxide, or thiourea derivative.

39. The antiviral composition of claim 35, wherein the antiretroviral therapy comprises combination antiretroviral therapy (cART).

40. The antiviral composition of claim 35, wherein the antiviral therapy comprises a nucleoside/nucleotide reverse transcriptase inhibitor, a non-nucleoside/nucleotide reverse transcriptase inhibitor, a protease inhibitor, an integrase strand transfer inhibitor, a fusion inhibitor, an entry inhibitor, a virus budding or maturation inhibitor, a polymerase inhibitor, a nonstructural protein 5A inhibitor, an RNA-dependent RNA polymerase inhibitor, a DNA polymerase inhibitor, or a capsid inhibitor, or any combination thereof.

41. The antiviral composition of claim 35, wherein the antiviral therapy comprises abacavir (ABC), didanosine (ddI), emtricitabine (FTC), lamivudine (3TC), stavudine (d4T), tenofovir (TFV, TFV-DP, TDF or TAF), zalcitabine (ddC), zidovudine (AZT), delavirdine (DLV), doravirine (DOR), efavirenz (EFV), etravirine (ETR), nevirapine (NVP), rilpivirine (RPV), MK-8507, elsulfavirine (VM1500), atazanavir (ATV), ATV/cobicistat (ATV/c), darunavir (DRV), darunavir/cobicistat (DRV/c), fosamprenavir (FPV), indinavir (IDV), lopinavir/ritonavir (LPV/r), nelfinavir (NFV), ritonavir (RTV), saquinavir (SQV), tipranavir (TPV), bictegravir (BIC), dolutegravir (DTG), elvitegravir (EVG), raltegravir (RAL), cabotegravir (CAB), enfuvirtide (T-20), T-1249, albuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, sifuvirtide, Sch-C, Sch-D, TAK-220, leronlimab (PRO-140), UK427857, maraviroc (MVC), aplaviroc, vicriviroc, cenicriviroc, adaptavir (RAP-101), nifeviroc (TD-0232), anti-GP120/CD4 or CCR5 bispecific antibodies, B-07, MB-66, polypeptide C25P, TD-0680, vMIP (Haimipu), a CXCR4 inhibitor, AMD-3100, a viral envelope adhesion inhibitor, a CD4 inhibitor, ibalizumab (IBA), temsavir (TMR), fostemsavir (FTR), UB-421, a viral gp120 inhibitor, a viral gp160 inhibitor, or a viral gp41 inhibitor, bevirimat (BVM), GSK3640254, GSK3739937, lenacapavir (LCV), PF74, GS-CA1, or any combination thereof.

42. The antiviral composition of claim 35, wherein the antiviral therapy comprises a broadly neutralizing antibody.

43. The antiviral composition of claim 35, wherein the broadly neutralizing antibody comprises VRC01, VRC07, 3BNC117, 10-1074, PGDM1400, 10E8, N6, 4/iMab, PGT121, elipovimab, N6LS, PGT-121, Elipovimab (GS-9722), Teropavimab (GS-5423), Zinlirvimab (GS-2872) or any combination thereof.
